(19) United States
(12) Patent Application Publication
(10) Pub. No.: US 2025/0195280 A1
(43) Pub. Date: Jun. 19, 2025

(54) FLUID MANAGEMENT LAYER FOR AN ABSORBENT ARTICLE

(71) Applicant: The Procter & Gamble Company, Cincinnati, OH (US)

(72) Inventors: Gerard Alain VIENS, Wyoming, OH (US); Misael Omar AVILES, Hamilton, OH (US); Olivia Maeve NEWMAN, Cincinnati, OH (US)

(21) Appl. No.: 18/978,101

(22) Filed: Dec. 12, 2024

Related U.S. Application Data
(60) Provisional application No. 63/610,672, filed on Dec. 15, 2023.

(51) Int. Cl. A61F 13/15; A61F 13/47; A61F 13/53; A61F 13/537; D04H 1/4258; D04H 1/4382

(52) U.S. Cl. CPC A61F 13/15203; A61F 13/47; A61F 13/53708; D04H 1/4258; D04H 1/43825; D04H 1/43835; A61F 2013/15406; A61F 2013/15447; A61F 2013/530233; A61F 2013/53024

(57) ABSTRACT

A fluid management layer that includes a nonwoven having a basis weight of from about 40 gsm to about 75 gsm, from about 15 to about 35 weight percent of cellulosic fibers, from about 65 to about 85 weight percent of bonding fibers, wherein the fluid management layer has a caliper factor of from about 0.26 to about 0.35, and wherein the cellulosic fibers and the bonding fibers have a decitex below about 2. The fluid management layer can be included in a disposable absorbent article.

### CROSS REFERENCE TO RELATED APPLICATIONS

This application claims the benefit, under 35 U.S.C. § 119(e), to U.S. Provisional Application No. 63/610,672 filed Dec. 15, 2023, the entire disclosure of which is fully incorporated by reference herein.

### FIELD OF THE INVENTION

The present disclosure generally relates to fluid management layer for a disposable absorbent article, in particular, a fluid management layer that is a needle punched nonwoven layer having improved performance characteristics.

### BACKGROUND OF THE INVENTION

Disposable absorbent articles such as feminine hygiene products, taped diapers, pant-type diapers and incontinence products are designed to absorb fluids from the wearer's body. Users of such disposable absorbent articles have several concerns. For example, leakage from products like catamenial pads, diapers, sanitary napkins, and incontinence pads is a significant concern.

Additionally, comfort and the feel of the product against the wearer's body is also a concern. To provide better comfort, current disposable absorbent articles are typically provided with a topsheet that is flexible, soft feeling, and non-irritating to the wearer's skin. The topsheet does not itself hold the discharged fluid. Instead, the topsheet is fluid-permeable to allow the fluids to flow into an absorbent core.

Regarding comfort, some consumers may desire a product that has sufficient thickness and stiffness to provide the desirable amount of protection while also being flexible. Lofty materials may be utilized to provide a thick cushiony feeling article. However, in use these lofty materials can experience various compressive loads. Recovery from these compressive loads is paramount in maintaining the cushiony feeling of the article. Exacerbating this issue is the fact that the characteristics of the materials of the absorbent article change once fluid is introduced into the article. Hence, an article that may meet a consumer's requisite criteria before use may no longer be comfortable, flexible, or have the desired stiffness to the user after a given amount of fluid has been absorbed by the absorbent article.

As such there is a need to create fluid management materials that have sufficient caliper and consumer desired recovery properties for use in absorbent articles while still delivering sufficient capillary action to facilitate fluid moving from the topsheet of the product to the absorbent core.

### SUMMARY OF THE INVENTION

Disclosed herein is a fluid management layer comprising: a nonwoven having a basis weight of from about 40 gsm to about 75 gsm, from about 15 to about 35 weight percent of cellulosic fibers, from about 65 to about 85 weight percent of bonding fibers, wherein the fluid management layer has a caliper factor of from about 0.26 to about 0.35, and wherein the cellulosic fibers and the bonding fibers have a decitex below about 2.

Also disclosed herein is a disposable absorbent article comprising a topsheet, a backsheet, an absorbent core disposed between the topsheet and the backsheet, and a fluid management disposed between the topsheet and the absorbent core wherein the fluid management layer comprises a nonwoven having a basis weight of from about 40 gsm to about 75 gsm, from about 15 to about 35 weight percent of cellulosic fibers, from about 65 to about 85 weight percent of bonding fibers, wherein the fluid management layer has a caliper factor of from about 0.26 to about 0.35, and wherein the cellulosic fibers and the polymeric fibers have a decitex below about 2.

### BRIEF DESCRIPTION OF THE DRAWINGS

FIG. 1 is a schematic representation of a disposable absorbent article constructed in accordance with the present disclosure;

FIG. 2 is a schematic representation of a disposable absorbent article constructed in accordance with the present disclosure;

FIG. 3 is fluid management layer top view (no cross lapping for comparison);

FIG. 4 is a fluid management layer top view with needlepunch;

FIG. 5 is a fluid management layer top view with needlepunch cross section with vertical needle bundle;

FIG. 6 is a fluid management layer top view with areas of capillary boosting points;

FIG. 7 is a schematic representation of asymmetric wings;

FIGS. 8-11 include equipment for Acquisition Time and Rewet Method; and

FIGS. 12-16 include views of equipment and graphs for the Bunch Compression & Bending Length test.

### DETAILED DESCRIPTION OF THE INVENTION

### Definitions

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene products, including feminine hygiene pads (also known as, for example, “sanitary napkins”, “menstrual pads”, “panty liners”, etc.).

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydroenhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of the fluid management layers described herein. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e., the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

“Lateral”—with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

“x-y plane,” with reference to an absorbent article, such as a feminine hygiene pad, or component thereof, when laid out flat on a horizontal surface, means any horizontal plane occupied by the horizontal surface or any layer of the article or component.

“z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a topsheet has a wearer-facing surface that lies closer to the wearer than the opposite, outward-facing surface of the topsheet.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Machine Direction” or “MD” as used herein with respect to an absorbent article such as a feminine hygiene pad or component thereof, refers to a direction parallel to the flow of the article or component through processing/manufacturing equipment.

“Cross Machine Direction” or “CD” as used herein with respect to an absorbent article such as a feminine hygiene pad or component thereof, refers to a direction perpendicular/orthogonal to the machine direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### General—Absorbent Article; Feminine Hygiene Pad

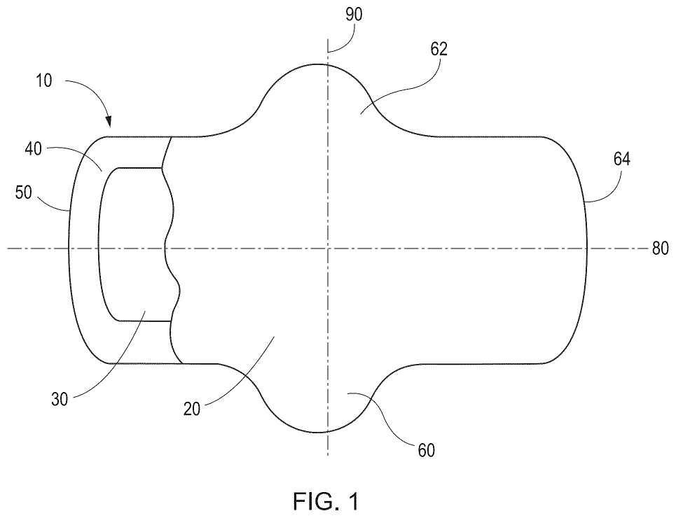

Referring to FIG. 1, an absorbent article as contemplated herein, such as a feminine hygiene pad 10, will include a wearer-facing surface and an opposing outward-facing surface. A liquid permeable topsheet 20 may form at least a portion of the wearer-facing surface and a liquid impermeable backsheet may form at least a portion of the outward-facing surface. An absorbent core including an absorbent structure 40 is disposed between the topsheet and the backsheet, and a fluid management layer 30 may be included and disposed between the absorbent structure 40 and the topsheet 20. (A fluid management layer as described herein is sometimes known in the art as an “acquisition/distribution layer” “distribution layer” or “secondary topsheet”, whose purpose is to dissipate energy from a fluid gush to the extent needed, provide a temporary volume of space for discharged fluid to occupy during the time required for an underlying absorbent structure to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants.

The topsheet 20 and the backsheet 50 may be joined together to form and define an outer periphery of the pad 10. The absorbent structure 40 and the fluid management layer 30 will each be sized to have outer perimeters disposed laterally and longitudinally inboard of the outer periphery. The absorbent structure 40 and the fluid management layer 30 may be dimensioned and shaped substantially similarly or identically to each other in the x-y directions, or they may have respective differing x-y dimensions and/or shapes. One or both may be manufactured to have a rectangular shape as suggested in FIG. 1, or one or both may be manufactured to have any other suitable shape, such as an oval shape, stadium shape, rounded rectangle shape, hourglass shape, peanut shape, etc. Shapes having concave profiles along the longitudinal edges may in some examples provide for enhanced comfort and/or conformity with the wearer's body.

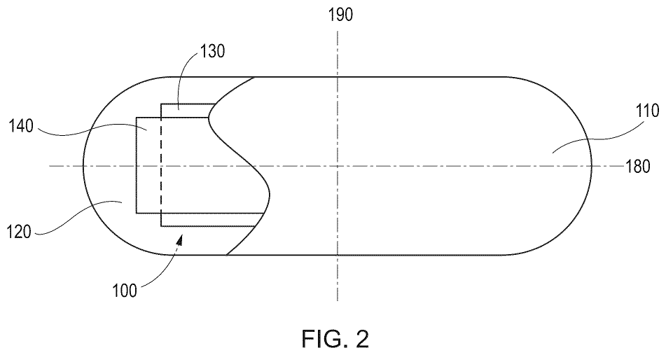

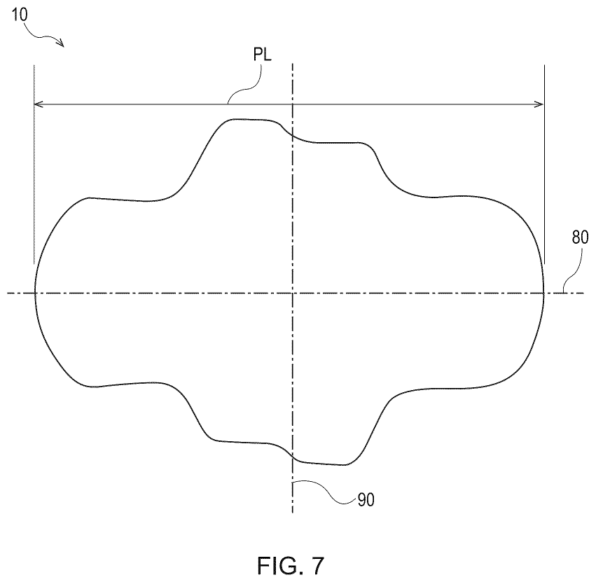

The topsheet 20 may be joined to the backsheet 50 by attachment any suitable attachment mechanism. The topsheet 20 and the backsheet 50 may be joined directly to each other in the article periphery, and may be indirectly joined together by directly joining them to the absorbent structure 40, the fluid management layer 30, and/or additional layers disposed between the topsheet 20 and the backsheet 50. This indirect or direct joining may be accomplished by any suitable attachment mechanism known in the art. Non-limiting examples of attachment mechanisms may include e.g., fusion bonds, ultrasonic bonds, pressure bonds, adhesive bonds, or any suitable combinations thereof. The absorbent article 10 may also comprise wings 60 extending outwardly with respect to a longitudinal axis 80 of the absorbent article 10. The absorbent article 10 may also comprise a lateral axis 90. As shown in FIG. 7 the wings may be asymmetric such as disclosed, for example, in U.S. patent publication numbers 2022/0409449, 2021/0307977, 2018/0325750, and 2018/0325751, which are all incorporated herein by reference. Referring to FIG. 2, the disposable absorbent article 100 having a topsheet 110, a backsheet 120, an absorbent core 130 disposed between the topsheet and the backsheet, and a fluid management layer 140 disposed between the topsheet and the absorbent core. The absorbent article 100 can have a fluid management layer 140 comprising a nonwoven having a basis weight of from about 40 gsm to about 75 gsm, from about 15 to about 35 weight percent of cellulosic fibers, from about 65 to about 85 weight percent of bonding fibers. The fluid management layer can have a caliper factor (mm per 10 gsm) of from about 0.26 to about 0.35, and the cellulosic fibers and the polymeric fibers can have a decitex below about 2. The absorbent article can have a Z-compression compression energy of from about 2.6 N·mm to about 4.0 N·mm, a 3 point MD bend dry bending stiffness of from about 15 N·mm\hat{ }2 and about 40 N·mm\hat{ }2, and a wet bunch compression % recovery of greater than about 40%. The absorbent article 100 can have a longitudinal axis 180. The absorbent article 100 may also comprise a lateral axis 190.

### Topsheet

### General

Generally, it is desirable that the topsheet 20 be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of a lotion composition onto an external or internal portion of a wearer's skin. The topsheet may comprise a nonwoven material.

Nonwoven fibrous topsheets 20 may be produced by any known procedure for making nonwoven webs, nonlimiting examples of which include spunbond processes, carding, wet-laid, air-laid, meltblowing processes, needle-punching, mechanical entangling, thermo-mechanical entangling, and hydroentangling.

Nonwoven materials suitable for use as a topsheet may include one strata of fibers or may be laminate of multiple nonwoven strata, which may comprise the same or different compositions (e.g., spunbond-meltblown laminate). In one specific example, the topsheet is a carded, air-through bonded nonwoven.

Topsheets contemplated herein do not include any predominant fraction of topsheet x-y surface area occupied by film. Some currently known topsheets for feminine hygiene pads include an apertured film, such as a hydroformed film or vacuum-formed film, alone or in combination with an adjacently-disposed nonwoven web material. The film may help to prevent liquids from resurfacing and contacting the wearer. The inventors have found, however, that a topsheet having the features described herein, particularly in combination with the fluid management layer described herein, can effectively prevent rewet to a comparable degree or better, than pads having topsheets comprising film across a predominant portion of topsheet x-y surface area. Without being bound by theory, it is believed that the careful selection of the fiber types in each of the strata in the fluid management layer, and the linear densities of the fiber types, can result in a desired combination of suitably fast acquisition, and low rewet, overcoming the typical tradeoff in these conflicting objectives associated with prior nonwoven topsheets. The improved performance is evident from the new combination of the unique nonwoven topsheet with a fluid management layer of the present disclosure.

### Basis Weight

The topsheet nonwoven may be manufactured to a basis weight of at least about 15 gsm, alternatively up to about 60 GSM specifically reciting all values within these ranges and any ranges created thereby. In some examples, a nonwoven topsheet contemplated herein may be manufactured to have a basis weight of about 15 gsm to 60 gsm, alternatively about 18 gsm to 40 gsm, and alternatively about 20 gsm to 30 gsm, specifically reciting all values within these ranges and any ranges created thereby. Suitable topsheet nonwoven may be manufactured to a basis weight of about 18 gsm to 40 gsm, alternatively from about 20 gsm to 30 gsm, alternatively from about 22 gsm to 26 gsm, specifically reciting all values within these ranges and any ranges created thereby. The range of desirable basis weight is influenced, at the lower end of the range, by the need for a level of web tensile strength needed for processing, and by consumer preferences for a level of opacity and substantiality of loft, feel and appearance. The range of desirable basis weight is influenced, at the upper end of the range, by the need for suitable rapid fluid acquisition and passage of fluid through the topsheet, and material cost concerns.

### Fiber Composition

Nonlimiting examples of woven and nonwoven materials suitable for use as the topsheet include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers. Synthetic fibers may include monocomponent fibers, bicomponent fibers or multicomponent fibers. (Herein, bi- or multicomponent fibers are fibers having cross sections divided into distinctly identifiable component sections each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven topsheets contemplated herein may include fibers having myriad combinations of constituent chemistries. For example, fibers may be spun from polymeric materials, such as polyethylene (PE) and/or polyethylene terephthalate (PET). Fibers may be spun in the form of bi-component fibers. In some examples, bi-component fibers may have a core component of a first polymer (for example, PET) in combination with another polymer as a sheath component, in a sheath-core bicomponent configuration. In more particular examples, PE may form the sheath component in combination with a PET core component. Fibers that include a PET component may be selected to help provide bulk and resilience and a resulting cushiony feel to the nonwoven web. Additionally, fibers that include a PET component, having resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component where sheath-core bi-component fibers are included. Additionally, without wishing to be bound by theory, it is believed that the use of polyethylene terephthalate as a core can help impart resilience to the topsheet.

Polyethylene, as a polymer component from which fibers may be spun, has a relatively lower melting temperature, and exhibits a relatively slick/silky surface feel as compared with other potentially useful polymers. PET has a relatively higher melting temperature, and exhibits relatively greater stiffness and resiliency. Accordingly, in some examples topsheet nonwoven fibers that are of a sheath-core bicomponent configuration may be desired, in which the sheath component is predominantly polyethylene, and the core component is predominantly PET. The polyethylene is useful for imparting the fibers and thus the topsheet with a silky feel, and for enabling inter-fiber bonding via heat treatment that cause sheaths of adjacent/contacting fibers to melt and fuse at the lower melting temperature of the polyethylene, while the PET is useful for imparting resilience, and does not melt in the heat treatment process. The inventors have found that a suitable weight ratio in such PE/PET sheath-core bicomponent fibers may be about 40:60 to about 60:40.

### Surface Treatment (Hydrophilicity/Hydrophobicity)

Depending upon the chemical composition thereof, surfaces of fibers will be, inherently, either hydrophilic or hydrophobic, to varying extents. For example, surfaces of fibers spun or otherwise formed from some types of polymers such as polyethylene and polypropylene will be, inherently, hydrophobic. In contrast, surfaces of other types of fibers, such as rayon fibers, will be inherently hydrophilic. Surfaces of natural fibers may be inherently hydrophilic or hydrophobic, but this may depend upon the processing the fibers have undergone. For example, cotton fibers as harvested bear coatings of natural oils and/or waxes and as such their surfaces are hydrophobic. After they have undergone processes including scouring and bleaching, however, the oils and/or waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, e.g., carding processes. These surface finishing agents or processing aids may be formulated to be either hydrophobic or hydrophilic, and to be substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes, and currently, several manufacturers in the nonwovens materials industry do this.

Noting that spun synthetic staple fibers may be obtained with either inherently hydrophobic or hydrophilic surfaces, or obtained with surface finishes that render their surfaces hydrophilic or hydrophobic at the purchaser's option, it may be desirable to choose fibers with surfaces that are either hydrophilic (“hydrophilic fibers”) or hydrophobic (“hydrophobic fibers”), or choose a blend of fibers of both types.

In some examples it may be preferable that the fiber constituents of the topsheet be, by weight, predominantly, substantially, or entirely hydrophobic, or rendered hydrophobic via fiber surface finish. A topsheet formed of a nonwoven web with predominately hydrophobic fiber constituents will be resistive to rewetting. On the other hand, if the sizes of the pores or inter-fiber voids within the fibrous structure of such nonwoven web are not sufficiently large, the topsheet may resist the passage of fluid from the wearing facing surface through to the absorbent core components of the article therebeneath, i.e., will not readily/rapidly acquire fluid, unless other features are included in combination, as described herein.

In other examples, fibers constituting portions, a majority (by surface area), or all, of the section of web material from which of the topsheet is formed, may be a blend of both hydrophobic fibers and hydrophilic fibers. In such examples, the hydrophilic fibers can serve to help wick fluid from the wearer-facing surface of the topsheet down to the absorbent core components beneath, while the hydrophobic fibers can serve to help the topsheet resist rewetting. The inventors have discovered that a successful balance may be struck for such examples.

Accordingly, in some examples the topsheet nonwoven may include a mix of hydrophobic and hydrophilic fibers. For example, the nonwoven may include at least about 40 percent, alternatively at least about 50 percent, or alternatively at least about 60 percent hydrophilic fibers by weight of the fibers, specifically including all values within these ranges and any ranges created thereby. In more particular examples, the nonwoven topsheet may comprise about 40 percent to 70 percent, alternatively about 45 percent to 68 percent, or alternatively from about 50 percent to 65 percent, by weight, hydrophilic fibers, specifically reciting all values within these ranges and any ranges created thereby. The topsheet nonwoven may include a blend of hydrophilic fibers and hydrophobic fibers in a weight ratio of hydrophilic fibers to hydrophobic fibers of 30:70 to 70:30, alternatively 35:65 to 65:35, and alternatively 40:60 to 60:40. As noted above, the hydrophilicity of the hydrophilic fibers may be effected by application of a surface treatment composition.

Without wishing to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

### Linear Density

Fibers are typically manufactured, selected and purchased by linear density specification, such expressed as denier or decitex. For fibers of a given polymer constitution, linear density correlates with fiber size/diameter.

In some examples, the fibers constituting the topsheet may be selected to have an average linear density of about 1.0 to 3.0 denier, alternatively about 1.5 to 2.5 denier, and alternatively about 1.8 to 2.2 denier, and all combinations of subranges within these ranges are contemplated herein. Fibers with varying linear densities within the ranges set forth above may be selected and included as well.

In other examples, the fibers constituting the topsheet may be selected to have an average linear density of about 3.0 to 5.0 denier, alternatively about 3.5 to 4.5 denier, and alternatively about 3.8 to 4.2 denier, and all combinations of subranges within these ranges are contemplated herein. It has been learned that fibers selected within these ranges, in combination with other features disclosed herein, may be deemed to constitute a topsheet material of acceptable softness to many consumers, as well as to provide other advantages over smaller fibers.

One advantage is that the relatively larger fibers generally provide a nonwoven web material with relatively larger inter-fiber/intra-web spaces or voids therewithin, thereby providing larger passageways through which fluid may more rapidly travel through the nonwoven from the wearer-facing side through to the outward-facing side (and thus to absorbent components below the topsheet). Additionally, although relatively larger fibers of a given composition are stiffer than smaller fibers of similar composition, which may somewhat compromise surface “softness” attributes, the greater fiber stiffness can also enhance a feeling of greater resiliency, springy or cushiony feel to the topsheet nonwoven.

### Staple Fiber Length

Suitable fibers may be staple fibers having a length of at least about 30 mm, 40 mm, or 50 mm, up to about 55 mm, or about 30 to 55 mm, or about 35 to 52 mm, reciting for said range every 1 mm increment therein. In particular example, staple fibers may have a length of about 38 mm.

### Applied Anti-Stick Agent

The absorbent article may include an anti-stick agent applied, to at least a portion of the wearer-facing surface of the topsheet, wherein the anti-stick agent includes a polypropylene glycol material. It is believed that an applied anti-stick agent as described herein serves functions that include reducing adherence of menstrual fluid to the user/wearer's skin, and facilitation of migration of menstrual fluid from the wearer-facing surface of the topsheet, down therethrough to the fluid management and/or absorbent structure layers beneath. Serving these functions can enhance user/wearer perceptions of cleanliness of her skin and of the topsheet, especially after repeated discharges of menstrual fluid. Examples of a suitable anti-stick agents and/or surfactants useful therein are disclosed in US 2009/0221978 (wherein the composition is called a “lotion”) and U.S. Pat. No. 8,178,748.

The anti-stick agent may include a polypropylene glycol (“PPG”) material. In some examples, the anti-stick agent may consist essentially of a polypropylene glycol material, including but not limited to polypropylene glycol homopolymer such as polypropylene glycol, and optionally, a carrier. In other examples, the anti-stick agent may include a polypropylene glycol material selected from the group of polypropylene glycol copolymer, polypropylene glycol surfactant, and mixtures thereof. The anti-stick agent including the polypropylene glycol material may serve to help reduce the adherence of menstrual fluid to the topsheet, and upon contact transfer of anti-stick agent to the user/wearer, reduce the adherence of fluid to her skin, thereby reducing staining on the topsheet and reducing soiling of the skin. The anti-stick agent may also help to improve continuous fluid acquisition of the absorbent article.

The anti-stick agent may be applied in any known manner, in any known pattern, and to the wearer-facing surface of the topsheet 20. For example, the anti-stick agent may be applied in a pattern of generally parallel, longitudinally- or laterally-oriented stripes or bands. To avoid compressing or displacing any portion of the topsheet nonwoven or any three-dimensional features thereof, it may be desired that the anti-stick agent be applied via spraying. The spray can be applied substantially uniformly.

The quantity of anti-stick agent applied may vary and can be adjusted for specific needs. For example, while not being bound by theory, it is believed that anti-stick agent may be applied at levels that are effective, of at least about 0.1 gsm, 0.5 gsm, 1 gsm, 2 gsm, 3 gsm, 4 gsm, 5 gsm, 10 gsm, up to about 15 gsm, or up to about 12 gsm, or up to about 10 gsm. It is believed that efficacy is not further enhanced above these upper limits, and so applications at basis weights exceeding these upper limits may be needless usage (waste) of anti-stick agent. The anti-stick agent can be applied within any subrange defined by any of the levels recited above (e.g., from about 0.1 gsm to about 15 gsm). These levels refer to the area of the topsheet surface to which the anti-stick agent is actually applied. The anti-stick agent can be applied on the majority, substantially all, or all of the surface area of the topsheet overlying the fluid management layer and/or absorbent core. This is because, as is believed, the anti-stick agent may enhance the ability of the topsheet to resist rewetting.

The anti-stick agent contemplated herein offers significant advantages over other anti-stick agents, including non-PPG derived surfactants and other surface modifying agents. The advantages may be deemed particularly useful for feminine hygiene pads. Without intending to be bound by theory, it is believed that the superior fluid handling properties of the PPG materials identified herein is a result of the way in which the PPG materials act on the solid components of menstrual fluid, as opposed to surface energy treatments which act on the water component of menses. Surface energy treatments may be less effective due to the presence of polar and dispersive components in menstrual fluid, which may inhibit the effectiveness of surface energy treatments. Because the PPG materials identified herein are typically not readily soluble in menstrual fluid, they can effectively coat surfaces without dissolving in the fluid, which provides a hydrated barrier whose electron donating dipoles repel negatively dipoled proteins, thereby rendering the menstrual fluid less apt to adhere to surfaces of the article or the wearer's skin. Less adherence of menstrual fluid to the wearer's skin and/or to the topsheet promotes better and faster fluid movement through the topsheet, and fewer, smaller and/or less visible stain patterns on used products.

The PPG materials identified herein can be applied as one component in an anti-stick agent, or can be applied neat (i.e., the anti-stick agent consists of PPG material). PPG materials, either neat and/or as part of an anti-stick agent, can be applied at varying quantity levels, depending on the fluid handling properties desired and desired treatment of the wearer's skin. PPG materials may be applied to the outer surface of the topsheet in any pattern, such as full coat, stripes or bands (oriented in the MD or CD direction), droplets, spiral patterns, and other patterns. An anti-stick agent including the PPG material may also be disposed near channels or embossed areas, when present.

The anti-stick agent contemplated herein may include a PPG material. PPG materials suitable for purposes contemplated herein include PPG homopolymer materials, PPG copolymer materials, and PPG surfactant materials, as well as mixtures thereof. The anti-stick agent may further comprise other optional ingredients. Suitable anti-stick agents include a PPG material, including but not limited to polypropylene glycol. Alternatively, the anti-stick agent comprises a PPG material selected from the group consisting of polypropylene glycol copolymer, polypropylene glycol surfactant, and mixtures thereof.

The anti-stick agents contemplated herein may include a PPG material at a level of about 0.1% to 100%, by weight of the anti-stick agent. In some examples, the anti-stick agent may include less than about 10%, alternatively from about 0.5% to 8%, and alternatively from about 1% to 5%, of a PPG material, by weight of the anti-stick agent. The anti-stick agent may include at least about 50%, alternatively from about 75% to 100%, and alternatively from about 90% to 100%, of a PPG material, by weight of the anti-stick agent.

Suitable PPG homopolymer materials may include those corresponding to the following formula:

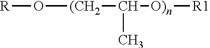

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R can be a hydrogen or methyl;
wherein R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R1 can be a hydrogen or methyl; and
wherein n is from 3 to 160, alternatively from 5 to 120, alternatively from 10 to 100, and alternatively from 20 to 80.

Optionally, the PPG homopolymer may include low level of glycerol or butanediol as part of its monomer raw material. If included, suitable ratios of glycerol or butanediol to propylene glycol may be about 1:1000 to about 1:2, alternatively from about 1:100 to about 1:5. The PPG homopolymer may have, but is not necessarily limited to, CAS Numbers 25322-69-4, 25791-96-2 and 25231-21-4.

Non-limiting examples of suitable PPG homopolymer materials include polypropylene glycol 4000 such as Pluriol P-4000 (BASF), Alkapol PPG-4000 (Alkaril Chemical) and Niax Polyol PPG 4025 (Union Carbide); polypropylene glycol 3500; polypropylene glycol 3000 such as Niax PPG 3025 (Union Carbide); polypropylene glycol 2000 such as Alkanol PPG-2000 (Alkaril Chemical) and Pluriol P-2000 (BASF), polypropylene glycol 1200 such as Alkapol PPG-1200 (Alkaril Chemical) and Glucam P-20 Humectant (Noveon); polypropylene glycol 1000 such as Niax PPG 1025 (Union Carbide); polypropylene glycol 600 such as Alkanol PPG-600 (Alkaril Chemical) and Glucam P-10 Humectant (Noveon); polypropylene glycol 400 such as Alkanol PPG-425 (Alkaril Chemical). polypropylene glycol 4000 glycerol ether such as Pluriol T-4000 (BASF); polypropylene glycol 2000 glycerol ether, polypropylene glycol 2000 butanediol ether, polypropylene glycol 1500 glycerol ether such as Pluriol T-1500 (BASF), polypropylene glycol 4000 with monomethyl ether, polypropylene glycol 2000 with dimethyl ether, polypropylene glycol 4000 with monobutyl ether, polypropylene glycol 2000 with monobuytyl ether, polypropylene glycol 1200 with dibutyl ether, polypropylene glycol 4000 with bis(2-aminopropyl ether), PPG-10 methyl glucose ether and PPG-20 methyl glucose either.

Suitable PPG homopolymer materials will typically have a number average molecular weight of about 400 to 10,000, alternatively about 600 to about 6,000, and alternatively from about 1,200 to about 4,800.

Suitable PPG copolymer materials include those in which the polypropylene glycol segments are present as an internal block component and/or as a terminal component, of the copolymer structure. The following formulae illustrate the internal block components and terminal block components:

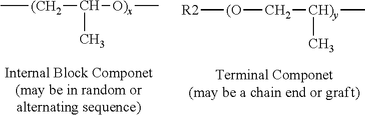

wherein x is 2 to 120, alternatively 2 to 80, and alternatively 3 to 60; y is 2 to 100, alternatively 2 to 50, and alternatively 3 to 30; R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate, and wherein R2 is hydrogen, methyl, ethyl, isopropyl or isobutyl.

Polymers suitable to form propoxylated copolymers with PPG for the present anti-stick agents include homopolymers of alkyl methicone, phenyl methicone, dimethicone, alkyl trimethicone, phenyl trimethicone, polyol, polyether (e.g., polyoxymethylene, polyoxyethylene and polyoxypropylene), polyimine, polyamide, polyacrylate, polyester, and copolymers containing one or multiple of these polymeric units. Non-limiting examples of suitable polymers include polydimethyl siloxane, polyethylimine, polyacrylic acid, poly(ethylene-co-acrylic acid), polymethacrylic acid, poly(ethylene-co-methacrylic acid), poly(vinyl acetate), polyvinylpyrrolidone, poly(ethylene-co-vinyl acetate), poly(butanediol), poly(neopentyl glycol), poly(ethylene adipate), poly(butylene adipate), poly(ethylene glutamate), poly(butylene glutamate), poly(ethylene sebacate), poly(butylene sebacate), poly(ethylene succinate), poly(butylene succinate), poly(ethylene terephthalate), poly(butylene terephthalate), polycaprolactone, and polyglycerol.

Non-limiting examples of suitable PPG copolymer materials include PPG-12 dimethicone such as Sisoft 910 (Momentive); bis-PPG-15 dimethicon/IPDI copolymer such as Polyderm-PPI-SI-WI (Alzo); PPG/polycaprolactone block copolymer; PPG/polybutanediol/PEG triblock copolymer; polyethylimine/PPG copolymer and polyacrylic acid-g-PPG graft copolymer.

Another suitable PPG material includes PPG surfactant materials. The following formula represents suitable PPG surfactant materials wherein the PPG segments constitute a part of the head functional group:

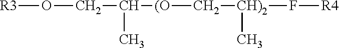

wherein R3 is hydrogen, alkyl, alkyl carbonyl, alkylenelamine, alkylenelamide, alkylene phosphate, alkylene carboxylic acid, alkylene sulfonate salt and alkylene quat with the maximum number of carbon element less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100, alternatively from 2 to 30, and alternatively from 3 to 25; and F is a functional group selected from the group of ether groups (including oxy, glyceryl, glucose, sorbitan, sucrose, monoethanolamine or diethanolamine), ester groups (including ester, glyceryl ester, glucose ester, sorbitan ester or sucrose ester), amine groups, amide groups, and phosphate ester groups.

The following formula represents suitable PPG surfactant materials wherein the PPG segments constitute an internal block group:

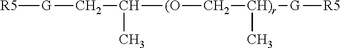

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120, alternatively from 4 to 50, and alternatively from 6 to 30; and G is ether, ester, amine, or amide linkage.

Non-limiting examples of suitable PPG surfactant materials include PPG-30 cetyl ether such as Hetoxol C30P (Global Seven); PPG-20 methyl glucose ether distearate such as Glucam P-20 Distearate Emollient (Noveon), PPG-20 methyl glucose ether acetate, PPG-20 sorbitan tristearate, PPG-20 methyl glucose ether distearate, PPG-20 distearate, PPG-15 stearyl ether such as Alamol-E (Croda-Unigema) and Procetyl 15 (Croda), PPG-15 stearyl ether benzoate, PPG-15 isohexadecyl ether, PPG-15 stearate, PPG-15 dicocoate, PPG-12 dilaurate, PPG-11 stearyl ether such as Varonic APS (Evonik); PPG-10 cetyl ether such as Procetyl 10 (Croda); PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether such as Promyristyl PM-3 (Croda), PPG-3 myristyl ether propionate such as Crodamol PMP (Croda), PPG-3 benzyl ether myristate such as Crodamol STS (Croda), PPG-3 hydrogenated castor oil such as Hetester HCP (Alzo), PPG-3-hydroxyethyl soyamide, PPG-2 Cocamide, PPG-2 lanolin alcohol ether and PPG-1 coconut fatty acid isopropanolamide such as Amizett IPC (Kawaken Fine Chemicals). Suitable PPG material is PPG-15 stearyl ether, such as the product sold as CETIOL E, by BASF Corporation (Florham Park, New Jersey, USA) and/or BASF SE (Ludwigshafen, Germany).

The anti-stick agents contemplated herein may include the carrier at a total carrier concentration ranging from about 60% to 99.9%, alternatively about 70% to 99.5%, alternatively about 80% to 99% by weight of the anti-stick agent.

Carriers suitable herein may include oils or fats such as natural oils or fats, or natural oil or fat derivatives, in particular of plant or animal origin. Non-limiting examples include avocado oil, apricot oil, apricot kernel oil, babassu oil, borage oil, borage seed oil, calendula oil, camellia oil, canola oil, carrot oil, cashew nut oil, castor oil, chamomile oil, cherry pit oil, chia oil, coconut oil, cod liver oil, corn oil, corn germ oil, cottonseed oil, eucalyptus oil, evening primrose oil, grape seed oil, hazelnut oil, jojoba oil, juniper oil, kernel oil, linseed oil, macadamia oil, meadowfoam seed oil, menhaden oil, mink oil, moringa oil, mortierella oil, olive oil, palm oil, palm kernel oil, peanut oil, peach kernel oil, rapeseed oil, rose hip oil, safflower oil, sandlewood oil, sesame oil, soybean oil, sunflower oil, sunflower seed oil, sweet almond oil, tall oil, tea tree oil, turnip seed oil, walnut oil, wheat germ oil, zadoary oil, or the hardened derivatives thereof. Hardened oils or fats from vegetal origin can include, e.g., hardened castor oil, peanut oil, soya oil, turnip seed oil, cottonseed oil, sunflower oil, palm oil, kernel oil, linseed oil, corn oil, olive oil, sesame oil, cocoa butter, shea butter and coconut oil.

Other non-limiting examples of fats and oils may include: butter, C12-C18 acid triglyceride, camellia oil, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, human placental lipids, hybrid safflower oil, hybrid sunflower seed oil, hydrogenated castor oil, hydrogenated castor oil laurate, hydrogenated coconut oil, hydrogenated cottonseed oil, hydrogenated C12-C18 triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lanolin and lanolin derivatives, lanolin alcohol, lard, lauric/palmitic/oleic triglyceride, lesquerella oil, maleated soybean oil, meadowfoam oil, neatsfoot oil, oleic/linoleic triglyceride, oleic/palmitic/lauric/myristic/linoleic triglyceride, oleostearine, olive husk oil, omental lipids, orange roughy oil, pengawar djambi oil, pentadesma butter, phospholipids, pistachio nut oil, placental lipids, rapeseed oil, rice bran oil, shark liver oil, shea butter, sphingolipids, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, wheat bran lipids, and the like, as well as mixtures thereof. Suitable carriers include caprylic/capric triglyceride. This material is currently available as, e.g., MYRITOL 318, a product of BASF Corporation (Florham Park, New Jersey, USA) and/or BASF SE (Ludwigshafen, Germany).

Other suitable carriers may include mono- or di-glycerides, such as those derived from saturated or unsaturated, linear or branch chained, substituted or unsubstituted fatty acids or fatty acid mixtures. Examples of mono- or diglycerides include mono- or di-C12-24 fatty acid glycerides, specifically mono- or di-C16-20 fatty acid glycerides, for example glyceryl monostearate, glyceryl distearate.

Carriers can also include esters of linear C6-C22-fatty acids with branched alcohols.

Carriers contemplated herein may also include sterols, phytosterols, and sterol derivatives. Sterols and sterol derivatives that can be used in the anti-stick agents of the invention include, but are not limited to: β-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

### Fluid Management Layer

The fluid management layer adds caliper to the absorbent article and is typically compressible, and resilient, which can impart a feeling of softness and/or a “cushiony” feel to the article. The absorbent articles contemplated herein exhibit good resiliency properties both in dry and wet conditions. The fluid management layer described herein has a nonwoven, cellulosic and bonding fibers and a caliper factor (mm per 10 gsm of basis weight) of from about 0.26 to about 0.35. The fibers have a decitex below about 2.

The fluid management layer may include a nonwoven having a basis weight of from about 40 to about 75 gsm. The fluid management layer can comprise any suitable shape including but not limited to oval, a stadium, rectangle, an asymmetric shape, peanut, trapezoid, rounded trapezoid, ovoid, nested and hourglass. In some examples, the fluid management layer may have a contoured shape, e.g., one that is narrower in the longitudinally intermediate region than in the end regions. In other examples, the fluid management layer may have a tapered shape that is a wider in one end region of the pad, and tapers to a narrower width in the other end region of the pad. The fluid management layer may have a long oval shape. The fluid management layer may have a nested shape where one end is concave, and the other end is convex.

In addition to the softness and resiliency benefits of compositions and structures for fluid management layers contemplated herein, stain size control and faster fluid acquisition may be obtained. Stain size is important in the way the absorbent article is perceived by the user. For feminine hygiene pads, when a stain visible on the pad after a duration of use/wear is relatively large along x-y directions, users may perceive that the pad is near failure based on the appearance of the stain and its proximity to the outer periphery of the pad. In contrast, a smaller, lighter stain can have a reassuring effect on the user/wearer, by creating a perception that the pad is not near failure because the edges of the stain lie substantially longitudinally and/or laterally short of the outer periphery of the pad.

Fluid acquisition speed of the pad may be deemed important to the user/wearer, as rapid acquisition can help make the user/wearer feel dry and clean. When the pad requires a relatively long time to drain discharged fluid from the topsheet, it can cause the user to feel wetness, and feel unclean.

To enable rapid acquisition/intake of discharged fluid, but also sufficient capillarity to dewater the topsheet, the fluid management layer has a particular pore volume range. The fluid management layer can have an average pore size of from about 90 to about 330 μm, alternatively the average pore size can be from about 100 to about 150 μm.

The fluid management layer can draw fluid through and from the topsheet via capillary action or wicking forces, of sufficient magnitude to overcome any resistance to passage of the fluid through the topsheet, or attraction the topsheet may have for the fluid, that may be present as a result of the composition and/or configuration of the topsheet. The fluid management layer also can accept and contain a gush of fluid by providing pore volume as a temporary reservoir, together with distribution functions, to efficiently utilize the absorbent structure, give it time to imbibe and absorb the fluid.

The inventors have found that to deliver the desired levels of softness and resiliency combined with the need for rapid acquisition and high capillarity needs, that the fluid management layer breaks technology limitations of the past. The fluid management layer of the present invention delivers high caliper, that is resilient and yet flexible combined with high capillarity and permeability.

Overall, the fluid management layers of the present disclosure may comprise from about 15 percent to about 35 percent by weight, from about 20 percent to about 35 percent by weight, from about 25 percent to about 35 percent by weight of the fluid management layer of cellulose, specifically including any values within these ranges and any ranges created thereby of cellulosic fibers. In one specific example, fluid management layers may comprise about 30 percent by weight of cellulosic fibers. Suitable cellulosic fibers include cotton, rayon, viscose, lyocell, natural cellulose, regenerated cellulose and combinations thereof. Particularly suitable cellulosic fibers include viscose. The cellulosic fibers have a decitex of below about 2; alternatively, from about 0.5 to about 1.7.

The cellulosic fibers of the fluid management layer may have any suitable cross-section profile shape (where the cross-section lies along a plane that is perpendicular with the greater length dimension of the fiber when it is straight). Some examples of suitable shapes may include trilobal, “H,” “Y,” “X,” “T,” round, or flat ribbon. Further, the absorbing fibers can have cross sections that are solid, hollow or multi-hollow. Other examples of suitable multi-lobed, cellulosic fibers for utilization in the fluid management layers described herein are disclosed in U.S. Pat. Nos. 6,333,108; 5,634,914; and 5,458,835. A trilobal fiber shape can improve wicking and improve opacity and stain concealment properties. Suitable trilobal rayon fibers are available from Kelheim Fibres GmbH (Kelheim, Germany) and sold under the trade name GALAXY. While each stratum may include a different shape of absorbing fiber, much like mentioned above, not all carding equipment may be suited to handle such variation between/among strata. In one specific example, the fluid management layer may include cellulosic fibers having a round (circular) shape.

The cellulosic fibers may include any suitable absorbent material. Suitable absorbent fibrous materials include cotton, cellulose (e.g., wood) pulp, regenerated cellulose (rayon, viscose, lyocell, etc.) or combinations thereof. In one example, the fluid management layer may include viscose fibers.

The staple length of the cellulosic fibers may be selected to be about 20 mm to 100 mm, or 30 mm to 50 mm, or even 35 mm to 45 mm, specifically reciting all values within these ranges and any ranges created thereby. In general, the fiber length of wood pulp is from about 4 to 6 mm and cannot used in conventional carding machines because the pulp fibers are too short. Accordingly, if wood pulp is desired as a fiber in the fluid management layer, additional processes to blend and add pulp to the carded webs may be beneficial. In some examples, pulp may be airlaid between carded webs with the combination being subsequently integrated. As another example, tissue made from pulp may be utilized in combination with the carded webs and the combination may be subsequently integrated.

Similarly, overall, the fluid management layers of the present disclosure may comprise from about 65 percent to about 85 percent by weight of the fluid management layer of bonding fibers, specifically reciting all values within these ranges and any ranges created thereby. Suitable bonding fibers include bicomponent polyethylene terephthalate/polyethylene, combinations of polyethylene, polypropylene, polyethylene terephthalate, Co-polyethylene terephthalate and combinations thereof. The bonding fiber can be polyethylene terephthalate/polyethylene wherein the core is polyethylene terephthalate, and the sheath is polyethylene. The bonding fibers can comprise bicomponent fibers. Particularly suitable bonding fibers can comprise polymeric fibers. The bonding fibers can have a decitex of less than about 2, alternatively from about 1 to about 2. The bonding fibers enhance the ability of the fluid management layer to recover its shape and/or caliper following application of compressive loads that are imposed during use.

And the fluid management layers of the present disclosure may also comprise from about 35 percent to about 55 weight percent of the fluid management layer of divider fibers. Suitable divider fibers include polypropylene, polyethylene terephthalate, bicomponent polyethylene, bicomponent polypropylene, bicomponent polyethylene terephthalate and combinations thereof. Suitable divider fibers have a decitex of from less than about 2, alternatively from about 0.5 to about 2. Particularly suitable divider fibers can comprise non-cylindrical polymeric fibers including but not limited to polypropylene. The divider fibers function to divide spaces in between the bonding and cellulosic fibers thereby creating smaller pore sizes that drive capillarity. The small size and optional non-cylindrical shape further accomplish this.

The cellulose, bonding and/or divider fibers can have a length of from about 10 to about 120 mm, alternatively from about 24 to about 95 mm, and alternatively from about 36 to about 75 mm. The fibers length can be selected from the same length, a different length or a combination thereof.

The weight fractions of cellulosic fibers, bonding fibers, and/or divider fibers may be determined via the Material Compositional Analysis method disclosed below.

In order to deliver the needed nonwoven caliper and the necessary pore structure for fluid performance it is necessary to utilize low decitex fibers as previously disclosed. It is additionally important to utilize a process that imparts material strength in both the MD and CD directions. This is particularly difficult as improper integration methods of the fibers will collapse the overall material caliper, especially since the fiber decitex are so low. The fluid management layer comprises integrated stitches at a stitch density of between 90 and 220 punches per square centimeter. The stitch direction is selected from the top, bottom and combinations thereof. Additionally, the fluid management layer has an MD:CD fiber orientation from about 1:1 to about 1:1.75.

The fluid management layer can have a MD peak load of from about 4 to about 85 Newtons and may also have a CD peak load of from about 4 to about 130 Newtons.

The fibers of the fluid management system can be from about 10 to about 120 mm in length, alternatively from about 24 to about 95 mm in length, and alternatively from about 36 to about 75 mm in length. The fibers length can be selected from a same length, a different length, or combinations of lengths.

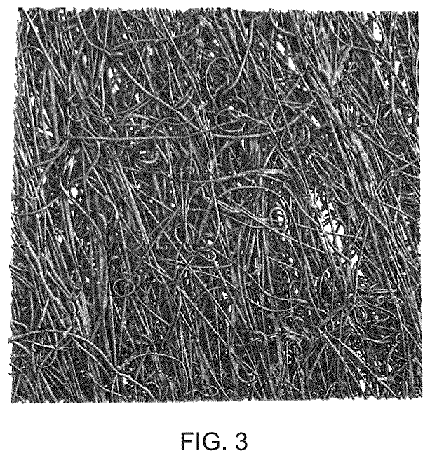

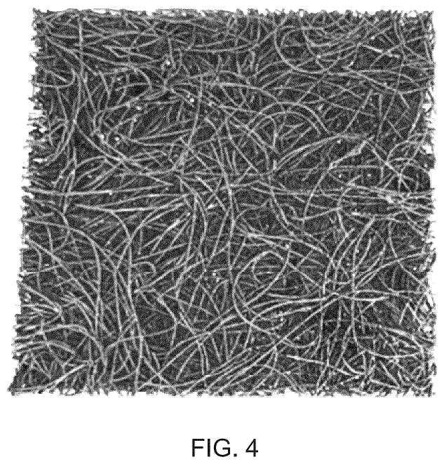

The inventors have found that the material can be both crosslapped and integrated via the use of specially crafted needles. This delivers the desired MD and CD material tensiles (reported as peak load) and MD/CD tensile ratios and maintains the overall material caliper as the energy to entangle is concentrated to specific fibers vs the entire web. Shown in FIG. 3 is a non-crosslapped fluid management layer. Shown in FIG. 4 is a crosslapped fluid management layer. In FIG. 4 the fibers are oriented in both the MD and CD directions.

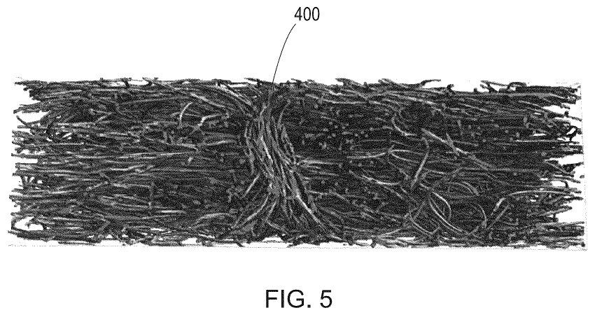

Needling reorients fibers from the x-y plane to the z-direction to create concentrated bundles of fibers oriented in the z-direction (As shown in FIG. 5). The vertical bundles create pathways for fluid to flow efficiently through the material in the z-direction to reach the core faster, particularly in gush situations. The vertical fiber bundles 400 also increase resilience and compression resistance in the z-direction.

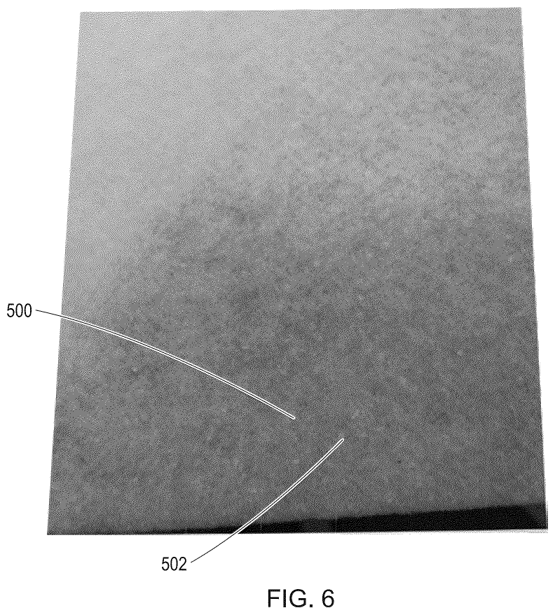

To further increase the ability of the fluid management layer to dry the topsheet, the fluid management layer can have small highly concentrated fiber areas spread throughout the fluid management layer. These small highly concentrated fiber areas, or capillary boosting points 500, 502 can be seen in FIG. 6. The capillarity boosting points have higher capillarity but as they are spread out over a wide area, they have minimal, or no, impact on overall permeability and hence minimal, or no, impact on the acquisition speed of the topsheet. These capillary boosting points can be spaced apart in various ways. For instance, there can be from about 1 capillary boosting point to about 10 capillary boosting points per square inch; alternatively, from about 3 to about 7 capillary boosting points per square inch. The capillary boosting points can be easily seen optically, especially with the use of a light table where the number of points is counted within a square inch of the material. The capillarity boosting points can vary in size from about 0.5 mm2 to about 5 mm2. The creation of the Capillary boosting points is controlled via careful calibration of the speeds within the carding unit and the speed of the needle punches combined with the small decitex fiber choices described herein.

The nonwoven of the present invention is initially fiber blended, accumulated and laydown and fed through one or more carding steps. The non-woven material is then cross-lapped prior to the web forming process step. Crosslapping is well known to a person skilled in the art. For example, the carded web material is moved forward and backwards when laid on a belt or carrier while its lower front portion is pulled perpendicular to this forward and backward movement whereby the web material overlaps in a z-like fashion. This imparts sufficient MD/CD tensile ratio.

The non-woven is then needlepunched, with the aid of one or more needle looms. Here a web of loose fibers, e.g., a web of carded fibers, is converted into a coherent non-woven fabric. The needlepunched fibers are mechanically oriented through the web. The needles can be arranged on a needle tool, e.g., a needle board or loom, in a non-lined arrangement. In the needle punching step at least one needle tool can be used in the range of from about 50 to 250, alternatively from about 70 to 200 and alternatively from about 90 to 180 needles per square inch.

The non-woven is then bonded via heat treatment after needling of the fluid management layer material. This bonding of the bonding fibers creates a support matrix which enhances resiliency and stiffness of the fluid management layer.

Absorbent articles that exhibit a soft cushiony feel, good resiliency and fluid handling characteristics are contemplated herein. Toward imparting these characteristics, the caliper of the fluid management layer may be deemed important. Typical calipers of webs from conventional spunlace lines achieve a caliper factor (caliper per 10 gsm of basis weight) of 0.03 to 0.12. In contrast, the fluid management layers contemplated herein can exhibit a caliper factor of at least 0.26. The fluid management layers contemplated herein can have a caliper factor of between 0.26 to about 0.35, including all values within these ranges and any ranges created thereby. It is important to note that the caliper factors mentioned heretofore are with regard to caliper obtained using the Caliper measurement method set forth below.

In the heat bonding process, the heating temperature selection may be impacted, in part, by the constituent composition(s) of the stiffening fibers, the design and operating parameters of the heating equipment, and the web processing speed (i.e., duration of exposure to the heated environment). To impart uniform stiffness across the fluid management layer, the heating equipment and operating parameters should be set up to provide uniform heating to the fluid management layer web. Even small variations in temperature can substantially impact the formation of fiber-to-fiber bonds between the bonding fibers and resulting tensile strength of the fluid management layer. An example of a suitable heat stiffening process that may be utilized is air-through heating, in which air heated to the selected heating temperature is blown and/or drawn (via vacuum) through the web along a direction that is approximately orthogonal to the larger planes defined by the web. Suitable MD/CD Peak Load ratios range from about 0.5 to 1.75.

Fluid management layers contemplated herein may be incorporated into a variety of absorbent articles. A non-limiting example of a schematic representation of an absorbent article in the form of a feminine hygiene pad as contemplated herein is shown in FIG. 1. As reflected, the pad 10 as contemplated herein may include a topsheet 20, a backsheet 50, and an absorbent structure 40 disposed between the topsheet 20 and the backsheet 50. A fluid management layer 30 may be disposed between the topsheet 20 and the absorbent structure 40. The pad has a wearer-facing surface 62 and an opposing outward-facing surface 64. The wearer-facing surface 62 is formed primarily by the topsheet 20 while the outward-facing surface 64 is formed primarily by the backsheet 50. Additional components (not shown) may be included proximate the wearer-facing surface 62 and/or the outward-facing surface 64. For example, if the absorbent article is an incontinence pad, a pair of barrier cuffs which extend generally parallel to a longitudinal axis of the pad 10 and may also form portions of the wearer-facing surface 62. Similarly, one or more deposits fastening adhesive (to be used by the user/wearer to affix the pad within her underwear, at an appropriate location, for use) may be present on the backsheet 50 and form a portion of the outward-facing surface 64 of the absorbent article.

### Caliper Factor

Unlike traditional spunlace materials, the fluid management layers can have a caliper factor (mm of caliper per 10 gsm) of at least about 0.26. The fluid management layer 30 can have a caliper factor of between 0.26 to about 0.35, including all values within these ranges and any ranges created thereby. The caliper and caliper factor of the fluid management layers of the present disclosure may be determined by the Caliper and Caliper Factor test methods disclosed herein.

### Basis Weight

The fluid management layers can have a basis weight of up to 75 grams per square meter (gsm); or a basis weight of up to 70 gsm; or a basis weight in the range of about 40 gsm to about 75 gsm; or in the range of about 50 gsm to about 70 gsm; or in the range of about 55 gsm to about 65 gsm, including any values within these ranges and any ranges created thereby. In another specific example, the fluid management layer 30 may have a basis weight of between 40 gsm to 60 gsm.

### Fibers

In order to enhance the stabilizing effect of the integration, crimped fibers may be utilized. As discussed in additional detail below, the fluid management layers of the present disclosure may comprise cellulose fibers, bonding fibers, and additionally may comprise divider fibers. One or more of these fibers may be crimped prior to integration. For example, where synthetic fibers are utilized, these fibers may be mechanically crimped via intermeshing teeth. And for the cellulosic fibers, these fibers may be mechanically crimped and/or may have a chemically induced crimp due to the variable skin thickness formed during creation of the cellulosic fibers.

### Absorbent Structure

The absorbent structure 40 of the present disclosure may have any suitable shape including but not limited to oval, a stadium, rectangle, an asymmetric shape, peanut, trapezoid, rounded trapezoid, ovoid, nested and hourglass. In some examples, absorbent structure 40 may have a contoured shape, e.g., one that is narrower in the longitudinally intermediate region than in the end regions. In other examples, the absorbent structure may have a tapered shape that is a wider in one end region of the pad, and tapers to a narrower width in the other end region of the pad. The absorbent structure may have a nested shape where one end is concave, and the other end is convex. The absorbent structure 40 may have varying stiffnesses in the MD and CD.

The configuration and construction of the absorbent structure 40 may vary (e.g., the absorbent structure 40 may have varying caliper zones, a hydrophilic gradient, a superabsorbent gradient, or lower average density and lower average basis weight acquisition zones). Further, the size and absorbent capacity of the absorbent structure 40 may also be varied to accommodate a variety of wearers. However, the total absorbent capacity of the absorbent structure 40 should be compatible with the design loading and the intended use of the disposable absorbent article or incontinence pad 10.

In some examples, the absorbent structure 40 may include a plurality of layers each having particular features and/or functions. Are some examples, the absorbent structure 40 may include a wrap (not shown) included to envelope enveloping the absorbent constituents of the absorbent structure. The wrap may be formed by one or more nonwoven materials, tissues, films or other materials, or laminates thereof. In one form, the wrap may be formed of only a single material, substrate, laminate, or other material that is wrapped at least partially around itself.

The absorbent structure 40 may include one or more adhesives, for example, to help immobilize the SAP or other absorbent materials within the first and second laminates.

Suitable absorbent structures comprising relatively high amounts of superabsorbent polymer (“SAP”—also known as “absorbent gelling material,” or “AGM”) with various core designs are disclosed in U.S. Pat. No. 5,599,335; EP 1 447 066; WO 95/11652; US 2008/0312622 A1; and WO 2012/052172

Additions to the absorbent structure are contemplated. Potential additions to the absorbent structure are described in U.S. Pat. Nos. 4,610,678; 4,673,402; 4,888,231; and 4,834,735. The absorbent structure may further include layers that mimic the dual core system containing an acquisition/distribution core of chemically stiffened fibers positioned over an absorbent storage core as described in U.S. Pat. No. 5,234,423; and in U.S. Pat. No. 5,147,345. These may be deemed useful to the extent they do not negate or conflict with the effects of the below described laminates of the absorbent structure of the present invention.

Some further examples of a suitable absorbent structures 40 that can be used in the absorbent article of the present disclosure are described in US 2018/0098893 and US 2018/0098891.

As noted above, absorbent articles including a fluid management layer contemplated herein may include a storage layer. Referring back to FIGS. 1A and 1B, a storage layer would generally be positioned at a location corresponding to that in which the absorbent structure 40 is depicted. The storage layer may be constructed as described regarding absorbent structures. The storage layer may contain conventional absorbent materials. In addition to conventional absorbent materials such as creped cellulose wadding, fluffed cellulose fibers, rayon or viscose fibers and comminuted wood pulp fibers (also known as airfelt or fluff pulp), and textile fibers, the storage layer may also include particles or fibers of superabsorbent material that imbibes fluids and forms hydrogels. (Such materials are also known as absorbent gelling materials (AGM).) AGM is typically capable of absorbing a relatively large weight quantity of body fluid per dry weight AGM, retaining it under moderate pressure. Synthetic fibers spun from polymers such as cellulose acetate, polyvinyl fluoride, polyvinylidene chloride, acrylics (such as ORLON), polyvinyl acetate, non-soluble polyvinyl alcohol, polyethylene, polypropylene, polyamides (such as nylon), polyesters, bi-component fibers, tricomponent fibers, mixtures thereof and the like can also be included in the secondary storage layer. The storage layer may also include filler materials, such as PERLITE, diatomaceous earth, VERMICULITE, or other suitable materials, that can serve to reduce changes of rewetting.

The storage layer or fluid storage layer may include absorbent gelling material (AGM) in a uniform distribution throughout or may include it in a non-uniform distribution. The AGM may be distributed and/or concentrated via deposit thereof into channels or pockets, or may be deposited in patterns including stripes, crisscross patterns, swirls, dots, or any other pattern, either two or three dimensional, that can be imagined. The AGM may be sandwiched between a pair of fibrous cover layers. AGM may be encapsulated, at least in part, by a single fibrous cover layer.

Portions of the storage layer may be formed substantially only of superabsorbent material/AGM, or may be formed of AGM distributed and dispersed in a suitable carrier structure such as a batt or accumulation of cellulose fibers in the form of fluff or stiffened fibers. One non-limiting example of a storage layer may include a first layer formed substantially only of AGM particles or fibers, that are placed or deposited onto a second layer that is formed of a distribution of AGM particles or fibers, within cellulose fibers.

Examples of absorbent structures formed of layers of superabsorbent material/AGM and/or layers of superabsorbent material/AGM dispersed within a batt or other accumulation of cellulose fibers, that may be utilized in the absorbent articles (e.g., sanitary napkins, incontinence products) contemplated herein are disclosed in US 2010/0228209A1. Absorbent structures comprising relatively high amounts of SAP/AGM with various core designs are disclosed in U.S. Pat. No. 5,599,335; EP 1 447 066; WO 95/11652; US. 2008/0312622 A1; WO 2012/052172; U.S. Pat. Nos. 8,466,336; and 9,693,910 to Carlucci. These may be used to configure the absorbent structure or storage layer.

### Backsheet

The backsheet 50 may be disposed beneath the absorbent structure 40 and be the outwardmost layer of the article, forming the outward-facing surface of the article. The backsheet 50 may be joined to the absorbent structure 40 and/or to the topsheet (about the outer periphery) by any suitable attachment methods known in the art. For example, the backsheet 50 may be secured to the absorbent structure 40 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet may be impervious, or substantially impervious, to liquids (e.g., urine, menstrual fluid) under ordinary conditions of use, and may be manufactured from a thin plastic film, although other flexible liquid impervious materials may also be used. The backsheet may prevent, or at least inhibit, exudates absorbed and contained in the absorbent structure from wetting underwear, outer clothing, bedding, etc. which may come into contact with or proximity to the article. However, in some examples the backsheet may be configured so as permit vapor to escape from the absorbent structure (i.e., is “breathable”) while in examples the backsheet may be configured so as to be vapor-impermeable (i.e., non-breathable). Backsheet may include a polymeric film such as a film of polyethylene or polypropylene. A suitable material for the backsheet is a thermoplastic film having a thickness of approximately 0.012 mm (0.5 mil) to 0.051 mm (2.0 mils), for example. Suitable materials for the backsheet film may have a basis weight of from about 8 to about 25 gsm. Any suitable liquid impermeable backsheet known in the art may be utilized with the present invention.

The backsheet serves as a barrier to prevent migration of fluids absorbed and retained in the absorbent structure, to the outward-facing surface of the pad. Suitable materials are soft, smooth, compliant, liquid and vapor pervious material that provides for softness and conformability for comfort, and is low noise producing so that movement does not cause unwanted sound.

Non-limiting examples of materials suitable for forming backsheets are described in U.S. Pat. Nos. 5,885,265; 6,462,251; 6,623,464; and 6,664,439. Examples of suitable dual- or multi-layer breathable backsheets include those described in U.S. Pat. Nos. 3,881,489; 4,341,216; 4,713,068; 4,818,600; EP 203 821; EP 710 471; EP 710 472; and EP 793 952. Additional examples of suitable single layer breathable backsheets for include those described in GB A 2184 389; GB A 2184 390; GB A 2184 391; U.S. Pat. Nos. 4,591,523; 3,989,867; 3,156,242; and WO 97/24097.

The backsheet may be a nonwoven web having a basis weight of about 20 gsm to 50 gsm. In one example, the backsheet may be a hydrophobic 23 gsm spunbond nonwoven web of 4 denier polypropylene fibers, available from Fiberweb Neuberger, under the trade designation F102301001. The backsheet may be coated with a non-soluble, liquid swellable material as described in U.S. Pat. No. 6,436,508.

The backsheet has an outward-facing side and an opposing wearer-facing side. The outward-facing side of the backsheet may include a non-adhesive area and an adhesive area. The adhesive area may be provided by any conventional means, for the purpose of enabling the user/wearer to affix the pad to the wearer-facing surface of her underwear at a location suitable for use. Pressure-sensitive adhesives have been found to work well for this purpose.

Traditional absorbent articles extend the width of the core in the crotch area as far as possible to acquire fluid that may escape to the edges. This can create an uncomfortable experience for the wearer. Other traditional absorbent articles narrow the core to improve comfort and attempt to extend the fluid management layers into wider areas in the crotch, but this does not create the necessary distance or changes in topography needed for best performance.

An additional feature of the absorbent article described herein is a stepped side barrier sufficient to capture fluid that may escape to the edges of the absorbent article. The disposable absorbent article comprises a topsheet, a backsheet, an absorbent core disposed between the topsheet and the backsheet, and a fluid management layer disposed between the topsheet and the absorbent core. The fluid management layer described herein has a basis weight of from about 40 gsm to about 75 gsm is combined with the topsheet in the process to create a differential tension composite web. This enables the topsheet to conform sufficiently around the side edges of the fluid management layer and then to be bonded to the backsheet while preserving the topographical features of the fluid management layer on the edges of the fluid management layer in the crotch area. The fluid management layer is wider than the absorbent core edge in the crotch area thereby creating a flexible and comfortable product while preserving a minimum of about 1.5 mm of step change in the crotch area. Suitable ranges for step change is from about 1.8 to about 3.5 mm.

### Test Methods

### Pore Volume Distribution Test Method

The Pore Volume Distribution Test Method is used to determine the mean absorption pressure, mean desorption pressure and mean pore size of a porous test specimen by measuring the associated fluid movement into and out of said specimen as stepped, controlled differential pressure is applied to the specimen in a test sample chamber.

### Method Principle

For uniform cylindrical pores, the radius of a pore is related to the differential pressure required to fill or empty the pore by the equation

$$Differential pressure = ( 2 γ cos Θ ) / r ,$$

where γ=liquid surface tension, Θ=contact angle, and r=pore radius.

Pores contained in natural and manufactured porous materials are often thought of in terms such as voids, holes or conduits, and these pores are generally not perfectly cylindrical nor all uniform. One can nonetheless use the above equation to relate differential pressure to an effective pore radius, and by monitoring liquid movement into or out of the material as a function of differential pressure characterize the effective pore radius distribution in a porous material. (Because nonuniform pores are approximated as uniform by the use of an effective pore radius, this general methodology may not produce results precisely in agreement with measurements of void dimensions obtained by other methods such as microscopy.)

The Pore Volume Distribution Test Method uses the above principle and is reduced to practice using the apparatus and approach described in “Liquid Porosimetry: New Methodology and Applications” by B. Miller and I. Tyomkin published in The Journal of Colloid and Interface Science (1994), volume 162, pages 163-170, incorporated herein by reference. This method relies on measuring the increment of liquid volume that enters or leaves a porous material as the differential air pressure is changed between ambient (“lab”) air pressure and a slightly elevated air pressure (positive differential pressure) surrounding the specimen in a sample test chamber. The specimen is introduced to the sample chamber dry, and the sample chamber is controlled at a positive differential pressure (relative to the lab) sufficient to prevent fluid uptake into the specimen after the fluid bridge is opened. After opening the fluid bridge, the differential air pressure is decreased in steps to 0, and in this process subpopulations of pores acquire liquid according to their effective pore radius. After reaching a minimal differential pressure at which the mass of fluid within the specimen is at a maximum, differential pressure is increased stepwise again toward the starting pressure, and the liquid is drained from the specimen. It is during this latter draining sequence (from minimal differential pressure, or largest corresponding effective pore radius, to the largest differential pressure, or smallest corresponding effective pore radius), that the fluid retention by the sample (g/g) at each differential pressure is determined in this method. After correcting for any fluid movement for each particular pressure step measured on the chamber while empty, the fluid retention by the sample (g/g) for each pressure step is determined via dividing the equilibrium quantity of retained liquid (g) associated with this particular step by the dry weight of the sample (g).

### Sample Conditioning and Specimen Preparation

The Pore Volume Distribution Test Method is conducted on samples that have been conditioned in a room at a temperature of 23° C.±2.0° C. and a relative humidity of 50%±5%, all tests are conducted under the same environmental conditions and in such conditioned room. Any damaged product or samples that have defects such as wrinkles, tears, holes, and similar are not tested. Samples conditioned as described herein are considered dry samples for purposes of this invention. Three specimens are measured for any given material being tested, and the results from those three replicates are averaged to give the final reported values. Each of the three replicate specimens has a diameter of 50 mm. The dry mass of each prepared test specimen is recorded to the nearest 0.001 g.

### Apparatus

Apparatus suitable for this method is described in: “Liquid Porosimetry: New Methodology and Applications” by B. Miller and I. Tyomkin published in The Journal of Colloid and Interface Science (1994), volume 162, pages 163-170. Further, any pressure control scheme capable of controlling the sample chamber pressure between 0 mm H₂O and 1200 mm H₂O differential pressure may be used in place of the pressure-control subsystem described in this reference. One example of suitable overall instrumentation and software is the TRI/Autoporosimeter (Textile Research Institute (TRI)/Princeton Inc. of Princeton, N.J., U.S.A.). The TRI/Autoporosimeter is an automated computer-controlled instrument for measuring pore volume distributions in porous materials (e.g., the volumes of different size pores within the range from 1 to 1000 μm effective pore radii). Computer programs such as Automated Instrument Software Releases 2000.1 or 2003.1/2005.1 or 2006.2; or Data Treatment Software Release 2000.1 (available from TRI Princeton Inc.), and spreadsheet programs may be used to capture and analyze the measured data.

### Method Procedure

The wetting liquid used is a degassed hexadecane (CAS 544-76-3, reagent grade, available from any convenient source). Liquid density is 0.773 g/cm³, surface tension 7 to be 27±1 mN/m, and the contact angle cos 0=1. A 90-mm diameter mixed-cellulose-ester filter membrane with a characteristic pore size of 1.2 μm (such Millipore Corporation of Bedford, MA, Catalogue #RAWP09025) is affixed to the porous frit (Monel plates with diameter of 90 mm, 6.4 mm thickness from Mott Corp., Farmington, CT, or equivalent) of the sample chamber.

Someone skilled in the art knows that it is critical to degas the test fluid as well as the frit/membrane/tubing system such that the system is free from air bubbles.

A metal weight weighing 414 g is placed on top of the sample to exert a constant confining pressure of 2.068 kPa during measurement.

The sequence of differential pressures that are run in the test, in mm H₂O, is as follows: 1100, 550, 367, 275, 220, 183, 138, 110, 92, 79, 69, 61, 55, 50, 46, 42, 39, 37, 34, 32, 31, 29, 28, 24, 22, 20, 18, 14, 9, 7, 6, 5, 4.5, 0, 4.5, 5, 6, 7, 9, 14, 18, 20, 22, 24, 28, 29, 31, 32, 34, 37, 39, 42, 46, 50, 55, 61, 69, 79, 92, 110, 138, 183, 220, 275, 367, 550, 1100.

The criterion for moving from one pressure step to the next is that fluid uptake/drainage from the specimen is measured to be less than 10 mg/min for 15 s.

A separate “blank” measurement is performed by following this method procedure on an empty sample chamber with no specimen or weight present on the membrane/frit assembly. Any fluid movement observed is recorded (g) at each of the pressure steps. Fluid retention data for a specimen are corrected for any fluid movement associated with the empty sample chamber by subtracting fluid retention values of this “blank” measurement from corresponding values in the measurement of the specimen.

Determination of Mean Absorption Pressure, Mean Desorption Pressure and Mean Pore Size As described above, the capillary fluid taken up (g) by the test specimen during the filling cycle (absorption) for each pressure step of differential pressure is corrected for any effect of the empty chamber and then divided by the dry mass of the specimen to arrive at capillary fluid uptake normalized by the dry sample mass recorded to the nearest 0.001 g/g. Likewise, the capillary fluid retained (g) by the test specimen during its drainage cycle for each pressure step of differential pressure is corrected for any effect of the empty chamber and then divided by the dry mass of the specimen to arrive at capillary fluid drainage normalized by the dry mass recorded to the nearest 0.001 g/g. The test specimen is considered to be 100% saturated at the lowest differential pressure, and at this pressure step the normalized uptake is at its maximum. Percent saturation for each pressure step of differential pressure is calculated by dividing the normalized uptake at each pressure step by the maximum normalized uptake and then dividing by 100. For the filling cycle (absorption), the differential pressure value at 50% saturation is recorded as the Mean Absorption Pressure (MAP) to the nearest 0.01 cm H2O. For the drainage cycle, the differential pressure value at 50% saturation is recorded as the Mean Desorption Pressure (MDP) to the nearest 0.01 cm H2O.

The effective pore radius, R, at each pressure step of differential pressure is calculated using the following equation and recorded to the nearest 0.01 micron.

$$Differential {pressure}_{i} = ( 2 γ cos Θ ) / {R}_{i}$$

where γ=liquid surface tension, Θ=contact angle, and r=pore radius.

The volume of fluid associated with each effective pore radius, V, is calculated using the following equation and the values are recorded to the nearest 0.01 mm³/g/micron.

$$V = \frac{\frac{( {w}_{i + 1} - {w}_{i} ) / d}{{R}_{i + 1} - {R}_{i}}}{{w}_{s a m p l e}}$$

where wᵢ is the fluid taken up (or retained, g), at pressure i (corrected for any effect of the empty chamber), d=fluid density (0.773 g/cm³), Rᵢ=pore radius at pressure i (microns) and wₛₐₘₚₗₑ=mass of the dry specimen (g).

The mean pore size of the test specimen for the filling cycle (absorption) is calculated by taking a weighted average of the pore size based on the volume of fluid using the following equation with the radius (R) and volume (V) values from the filling cycle (absorption).

$$W = \frac{{\sum }_{i = 1}^{n} {w}_{i} {X}_{i}}{{\sum }_{i = 1}^{n} {w}_{i}}$$

where W=weighted average of the pore size, n=number of items to be averaged, wᵢ=volume of fluid, V, and Xᵢ=effective pore radius, R. The mean pore size for absorption is recorded to the nearest 0.1 micron.

Likewise, the mean pore size of the test specimen for the drainage cycle is calculated using the same equation with the radius and volume values from the drainage cycle. The mean pore size for drainage is recorded to the nearest 0.1 micron.

In like fashion, repeat the entire procedure for a total of three replicate test specimens. The arithmetic mean of the three values for each recorded parameter (MAP, MDP, mean pore size for absorption and mean pore size for drainage) is reported.

### Acquisition Time and Rewet Method (ATRM)

This method describes how to measure gush acquisition time, interfacial free fluid amount as well as low and high pressure rewet values for an absorbent article loaded with new Artificial Menstrual Fluid (nAMF; preparation provided separately herein). A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low and high pressure rewet are measured after the last liquid dose. Surface Free Fluid (SFF) is the amount of fluid that remains in the topshseet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2 C° and 50%±2% relative humidity.

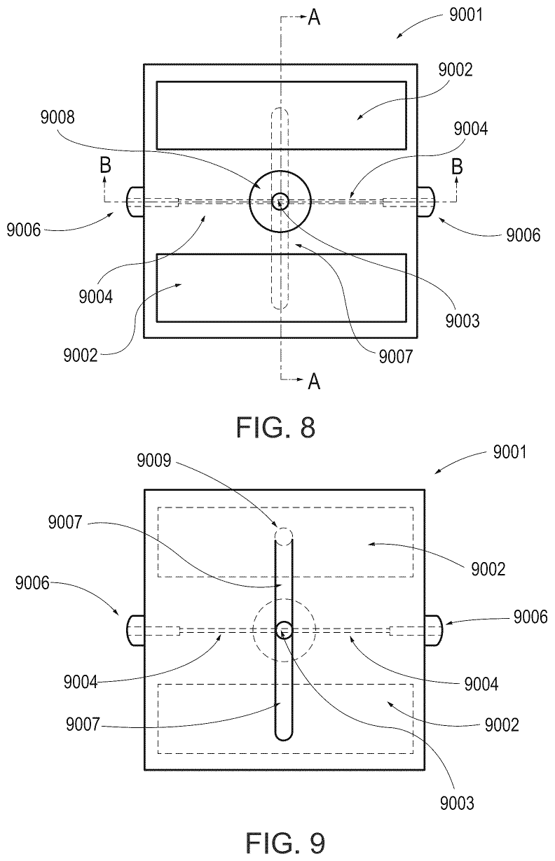

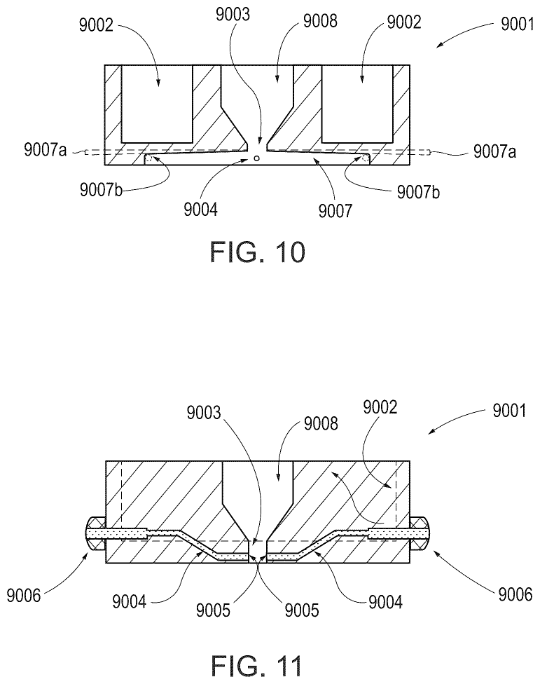

Referring to FIGS. 8 through 11, the strikethrough plate 9001 is constructed of Plexiglas, or equivalent, with an overall dimension of 10.2 cm long by 10.2 cm wide by 3.1 cm tall. A central, test fluid well 9008 has a circular opening of 25 mm in diameter is located at the top plane of the plate with initial lateral walls that extend 15 mm deep at a 90° angle and then slope downward at an angle of 82° for an additional depth of 7.5 mm to reach the test fluid reservoir 9003. The test fluid reservoir 9003 is concentric to the test fluid well 9008 and has a diameter of 6.6 mm with lateral walls that extend 5 mm deep at a 90° angle. The test fluid reservoir 9003 opens into the longitudinal fluid channel 9007 located at the bottom of the plate. The longitudinal fluid channel 9007 has lateral walls that initially extend 3.5 mm deep at the midpoint of the channel (just beneath the test fluid reservoir 9003), then slant downward at an angle 9007a of 0.72° towards each longitudinal end of the channel to a final depth of 3 mm. The longitudinal fluid channel opens to the bottom plane of the plate for the fluid to be introduced onto the underlying test sample. The longitudinal fluid channel 9007 is centered over the test fluid reservoir 9003 and extends in a direction that is perpendicular to the electrodes 9004. The longitudinal fluid channel 9007 has a width of 5 mm and a length of 80 mm, with lateral edges that are rounded with a radius 9007b of 1.0 mm. The longitudinal ends of the longitudinal fluid channel 9007 are rounded with a radius 9009 of 2.5 mm. Two wells 9002 (80.5 mm long by 24.5 mm wide by 25 mm deep) located outboard of the fluid reservoir, are filled with stainless steel shot (or equivalent) to adjust the overall mass of the plate to provide a constraining pressure of 0.10 psi (7.0 g/cm²) to the Test Area. The procedure for determining the test area is subsequently described herein. Electrodes 9004 are embedded in the plate 9001, connecting the exterior banana jacks 9006 to the inside wall 9005 of the longitudinal fluid channel 9003. A circuit interval timer is plugged into the jacks 9006, monitors the impedance between the two electrodes 9004, and measures the time from introduction of the nAMF into reservoir 9003 until the nAMF drains from the reservoir. The timer has a resolution of 0.01 sec.

A pretreatment plate is used in combination with a pretreatment weight to apply tiny droplets of nAMF to the surface of the test sample as a means to prime the surface of the test sample prior to the introduction of the full liquid dose. The pretreatment plate is constructed of Plexiglass, or equivalent, that is 14 inch (35.6 cm) long by 8 inch (20.3 cm) wide with a thickness of about 0.25 inch (6.4 mm). The pretreatment plate has five circular markers, each 5 mm in diameter, placed 1 cm apart (center to center) that are aligned along the longitudinal axis of the plate. The central marker is centered at the lateral midpoint of the plate. These markers indicate the placement of the nAMF droplets. The markers are located on the underside of the pretreatment plate and can be milled out or simply drawn on with a permanent marker, or equivalent. The pretreatment weight is 10.2 cm×10.2 cm and consists of a flat, smooth rigid material (e.g., stainless steel) with an optional handle. The pretreatment weight (including optional handle) has a total mass of 726 g+0.5 g to give a pressure of 0.10 psi (7.0 g/cm²) across the bottom surface area of the weight.

When measuring the interfacial fluid amounts, a rubber pad is used to provide a reproducibly flat surface that enables even pressure distribution. The IFF rubber pad is constructed from high strength neoprene rubber with 40 A durometer and a thickness of ⅛ inch (available from W.W. Grainger, Inc, item #1DUV4, or equivalent) and cut to dimensions of 6 inch (15.2 cm) by 6 inch (15.2 cm).

For the overall rewet portion of the test, a padded weight assembly that applies 0.5 psi (35.1 g/cm²) to the Test Area is required. The procedure for determining the test area is subsequently described herein. The rewet weight is constructed as follows. Lay a piece of polyethylene film (about 25 microns thick, any convenient source) horizontally flat on a rigid bench surface. A piece of polyurethane foam (25 mm thick, density of 1.0 lb/ft³, IDL 24 psi, available from Concord-Renn Co. Cincinnati, OH, or equivalent) is cut to 10.2 cm by 10.2 cm and then laid centered on top of the film. A piece of Plexiglas (10.2 cm by 10.2 cm and about 6.4 mm thick) is then stacked on top of the polyurethane foam. Next the polyethylene film is used to wrap the polyurethane foam and Plexiglas plate securing it with transparent tape. A metal weight with handle is stacked on top of, and fastened to, the Plexiglass plate such that the total mass of the padded weight assembly can be adjusted to apply a pressure of 0.5 psi (35.1 g/cm²) to the Test Area.

For the IFF, SFF and overall rewet steps, various layers of filter paper are required. The filter paper is conditioned at 23° C.±2° C. and 50%±2% relative humidity for at least 2 hours prior to testing. A suitable filter paper has a basis weight of about 88 gsm, a thickness of about 249 microns with an absorption rate of about 5 seconds, and is available from Ahlstrom-Munksjo (Mt. Holly Springs, PA) as grade 632, or equivalent. The filter paper has dimensions of 5 inch by 5 inch (12.7 cm by 12.7 cm).

Test samples are conditioned at 23° C.±2° C. and 50%±2% relative humidity for at least 2 hours prior to testing. Test samples are removed from their outer packaging and the wrappers are opened to unfold the product, if applicable, using care not to press down or pull on the products while handling. No attempt is made to smooth out wrinkles. Tear the release paper between the wings, if applicable, and lay the sample on a horizontally flat, rigid surface with the body-side facing up (e.g., panty-side down). Determine the dose location as follows. For symmetrical products (i.e., the front of the product is the same shape and size as the back of the product when laterally divided along the midpoint of the longitudinal axis of the product), the dose location is the intersection of the midpoints of the longitudinal and lateral axes of the absorbent core. For asymmetrical products (i.e., the front of the product is not the same shape and size as the back of the product when laterally divided along the midpoint of the longitudinal axis of the product), the dose location is the midpoint of the product's wings at the lateral midpoint of the absorbent core. For products that have a foam core with holes and slits either punched out or printed, the dose location is the longitudinal midpoint of the hole-punched (or hole-printed) region at the lateral midpoint of the absorbent core. Once determined, mark the dose location with a small dot using a black, fine-tip, permanent marker. If wings are present, fold them to the back of the product.

Determine the Test Area of the test sample, as follows. This area will be used so that the mass of the strikethrough plate and the mass of the rewet weight can be properly adjusted to deliver the required pressure (0.1 psi and 0.5 psi, respectively). Measure the width of the absorbent core of the test sample as the distance between one lateral edge of the core to the other lateral edge of the core along a line that is positioned at the dosing location and runs perpendicular to the longitudinal axis of the test sample, and record as core width to the nearest 0.01 cm. Now multiply the core width by 10.2 cm (the length of the strikethrough plate and rewet weight) and record as Test Area to the nearest 0.1 cm². The total mass of the strikethrough plate is the Test Area multiplied by 7 g/cm². The total mass of the rewet weight is the Test Area multiplied by 35.1 g/cm².

The test sample is pretreated with nAMF as follows. Place the pretreatment plate onto a horizontally flat, rigid surface such that the side with the circular markers is facing down. Using a single channel, fixed volume pipettor, accurately dispense 50 μL of nAMF onto the topside of the pretreatment plate at the location of each of the five circular markers. Position the test sample above the pretreatment plate such that the body-side of the sample is facing the plate, the longitudinal axis of the sample and plate are aligned, and the pre-marked dose location on the test sample is centered over the central droplet of nAMF on the pretreatment plate. After properly positioned, place the test sample into contact with the pretreatment plate, then immediately apply the pretreatment weight onto the back side of the test sample, centering it over the dose location/central droplet of nAMF on the pretreatment plate. Start a 40 second timer. After 40 seconds have elapsed, remove the pretreatment weight from the test sample and remove the test sample from the pretreatment plate. Invert the test sample so that the body-side is facing up, place it onto a horizontally flat, rigid surface and immediately proceed with the steps that follow.

The first acquisition time (ACQ-1) is measured as follows. Connect the electronic circuit interval timer to the strikethrough plate 9001 and zero the timer. Position the strikethrough plate 9001 above the body-side of the test sample such that the long axis of the longitudinal fluid channel 9007 on the underside of the strikethrough plate 9001 is aligned with the longitudinal axis of the test sample, and ensure that the fluid reservoir 9003 is centered over the pre-marked dose location on the test sample. To note, nAMF should be visible through the fluid reservoir 9003 at the dose location on the test sample. After properly positioned, gently place the strikethrough plate 9001 onto the test sample. Using an adjustable volume pipettor, accurately dispense 2.0 mL of nAMF into the fluid well 9008 in the strikethrough plate 9001. The fluid is dispensed, without splashing, along the angled walls of the fluid well 9008 within a period of 3 seconds or less. Immediately after the fluid has been acquired, record the first acquisition time (ACQ-1) displayed on the circuit interval timer to the nearest 0.1 seconds. Leave the strikethrough plate 9001 in place on the test sample, and immediately start a 2 minute timer.

After 2 minutes have elapsed, measure the first Interfacial Free Fluid (IFF-1) as follows. Place the IFF rubber pad onto a horizontally flat, rigid surface. Measure the mass of one layer of filter paper to the nearest 0.0001 g and record as IFF-1ᵢₙᵢₜᵢₐₗ. Place the filter paper centered onto the IFF rubber pad. Transfer the strikethrough plate 9001 from the test sample to the pre-weighed filter paper such that the plate is centered on the filter paper, and immediately start an 8 minute timer. After 10 seconds have elapsed on the 8 minute timer, remove the strikethrough plate from the filter paper and gently replace it back onto the test sample, exactly as previously positioned. Within the next 10 seconds, measure the mass of the filter paper to the nearest 0.0001 g and record as IFF-1_{final}.

The second acquisition time (ACQ-2) is measured as follows. After 8 minutes have elapsed, apply the second gush of fluid using an adjustable volume pipettor to accurately dispense 4.0 mL of nAMF into the fluid well 9008 in the strikethrough plate 9001, as previously described. Immediately after the fluid has been acquired, record the second acquisition time (ACQ-2) displayed on the circuit interval timer to the nearest 0.1 second. Leave the strikethrough plate 9001 in place on the test sample, and immediately start a 2 minute timer.

After 2 minutes have elapsed, measure the second Interfacial Free Fluid (IFF-2) as follows. Place the IFF rubber pad onto a horizontally flat, rigid surface. Measure the mass of a fresh, single layer of filter paper to the nearest 0.0001 g and record as IFF-2ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and gently replace it back onto the test sample, exactly as previously positioned. Within the next 10 seconds, measure the mass of the filter paper to the nearest 0.0001 g and record as IFF-2_{final}.

The third acquisition time (ACQ-3) is measured as follows. After 8 minutes have elapsed, apply the third gush of fluid using an adjustable volume pipettor to accurately dispense 2.0 mL of nAMF into the fluid well 9008 in the strikethrough plate 9001, as previously described. Immediately after the fluid has been acquired, record the third acquisition time (ACQ-3) displayed on the circuit interval timer to the nearest 0.1 second. Leave the strikethrough plate 9001 in place on the test sample, and immediately start a 2 minute timer.

After 2 minutes have elapsed, measure the third Interfacial Free Fluid (IFF-3) as follows. Place the IFF rubber pad onto a horizontally flat, rigid surface. Measure the mass of a fresh, single layer of filter paper to the nearest 0.0001 g and record as IFF-3ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and set it on its side so that the pad-side of the plate is not contacting the bench. Within the next 10 seconds, measure the mass of the filter paper to the nearest 0.0001 g and record as IFF-3_{final}.

Measure Surface Free Fluid (SFF) as follows. After 8 minutes have elapsed, measure the mass of a fresh stack of 5 filter papers to the nearest 0.0001 g and record as SFFᵢₙᵢₜᵢₐₗ. Place the stack of filter papers on top of the body-side of the test sample such that they are centered over the dose location. Now gently place the strikethrough plate 9001 on top of the filter papers such that the pad-side of the plate is centered on the filter papers, and immediately start a 10 second timer.

After 10 seconds have elapsed, remove the strikethrough plate 9001 from the filter papers and set it aside. Measure the mass of the stack of 5 filter papers to the nearest 0.0001 g and record as SFF_{final}. Immediately proceed to the next step.

Measure overall rewet as follows. Measure the mass of a fresh stack of 5 filter papers to the nearest 0.0001 g and record as REWETᵢₙᵢₜᵢₐₗ. Place the filter papers on top of the body-side of the test sample such that they are centered over the dose location. Now place the padded Rewet Weight on top of the stack of filter papers such that the weight is centered on the filter paper stack, and immediately start a 30 second timer. After 30 seconds have elapsed, remove the rewet weight and measure the mass of the stack of 5 filter papers to the nearest 0.0001 g, then record as REWET_{final}. Discard the sample and thoroughly clean and then dry the fluid well 9008, fluid reservoir 9003, longitudinal fluid channel 9007 and the bottom surface of the strikethrough plate 9001 prior to testing the next sample.

Make the following calculations for each of the parameters measured, as follows. Calculate Total Gush Absorbency Time as the sum of ACQ-1, ACQ-2 and ACQ-3, and record to the nearest 0.1 second. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate Total IFF as the sum of IFF-1, IFF-2 and IFF-3, and record to the nearest 0.1 g. Calculate SFF by subtracting SFFᵢₙᵢₜᵢₐₗ from SFF_{final}, and record to the nearest 0.0001 g. Calculate Total IFF+SFF as the sum of Total IFF and SFF, and record to the nearest 0.1 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the arithmetic mean of the three individually recorded measurements for each Acquisition Time (ACQ-1, ACQ-2 and ACQ-3) to the nearest 0.1 seconds, Total Gush Absorbency Time to the nearest 0.1 seconds, Interfacial Free Fluid (IFF-1, IFF-2 and IFF-3) to the nearest 0.0001 g, Total IFF to the nearest 0.1 g, Surface Free Fluid (SFF) to the nearest 0.0001 g, Total IFF+SFF to the nearest 0.1 g, and Overall Rewet to the nearest 0.0001 g.

### Fiber Decitex (Dtex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning Calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10 \; 000 m \times {a}_{k} \times {ρ}_{k} \times 1 {0}^{- 6}$$

where dₖ is in units of grams (per calculated 10,000 meter length), aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

### Basis Weight

The basis weight of a test sample is the mass (in grams) per unit area (in square meters) of a single layer of material and is measured in accordance with compendial method WSP 130.1. The mass of the test sample is cut to a known area, and the mass of the sample is determined using an analytical balance accurate to 0.0001 grams. All measurements are performed in a laboratory maintained at 23° C.±2° C. and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Air Permeability

The measurements for air permeability provided herein were obtained by using Worldwide Strategic Partners (WSP) Test Method 70.1.

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it is larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously is the caliper per 10 gsm of basis weight of the sample. So, the equation is caliper/(basis weight/10).

### Density

Density is calculated based upon the basis weight and caliper with appropriate unit conversion to arrive at g/cc.

### Material Compositional Analysis

The quantitative chemical composition of a test specimen comprising a mixture of fiber types is determined using ISO 1833-1. All measurements are performed in a laboratory maintained at 23° C.±2° C. and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Compression Caliper

The caliper, or thickness, of a test specimen is measured as the distance between a reference platform on which the specimen rests and a pressure foot that exerts a specified amount of pressure onto the specimen over a specified amount of time. For this method, a series of pressures are applied to the test specimen for a specified time, with a recovery period in between. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure onto the test specimen at each of the specified pressures (+0.01 kPa) in the stepped pressure series 0.50, 1.00, 2.00, 3.00, 5.00, and 0.50 kPa. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has an area of 25 cm², however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen from a sample of the material being evaluated. The test specimen is obtained from an area free of folds or wrinkles, and it is larger than the pressure foot.

To measure caliper, first ensure the pressure to be exerted onto the sample is adjusted to the first pressure in the stepped pressure series, 0.50 kPa. Now zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 4 seconds, then record the caliper of the test specimen to the nearest 0.001 mm noting the test pressure applied. Remove the pressure from the test specimen and set a 30 second timer accurate to 0.1 seconds (any convenient source). Now adjust the pressure to the next pressure setting in the stepped series, 1.00 kPa, and zero the micrometer against the horizontal flat reference platform. After 30 seconds have elapsed, place the test specimen onto the platform with the same test location centered below the pressure foot. In like fashion, lower the pressure foot and record the caliper of the test specimen to the nearest 0.001 mm noting the pressure applied. Remove the pressure from the test specimen and set a 30 second timer. Repeat this entire process for each of the pressures in the stepped pressure series, in the order as follows: 0.50, 1.00, 2.00, 3.00, 5.00, 0.50 kPa. Record the caliper to the nearest 0.001 mm for each pressure, noting the pressure applied. For the 0.50 kPa pressure setting, note 0.50 kPa Initial and 0.50 kPa Final, respectively, along with the caliper. The caliper foot is to be applied to the same test location on the test specimen for each pressure applied.

In like fashion, repeat for a total of three replicate test specimens. Calculate the arithmetic mean for the caliper measurements at each pressure, and report as Caliper to the nearest 0.001 mm, noting the pressure applied for each, along with Initial and Final for the 0.50 kPa setting. From these results, Caliper Decrease can be calculated between any of the pressure settings used by simply subtracting the caliper obtained at a higher pressure from the caliper obtained at a lower pressure, and reporting to the nearest 0.001 mm.

### Repetitive Acquisition Time and Rewet (RATR)

Acquisition time is measured for an absorbent article dosed with Artificial Menstrual Fluid (AMF) as described herein, using a strikethrough plate and an electronic circuit interval timer. The time required for the absorbent article to acquire a series of doses of AMF is recorded. Subsequent to the acquisition test, a rewet test is performed. All measurements are performed in a laboratory maintained at 23° C.±2° C. and 50%±2% relative humidity.

Referring to FIGS. 4-6B, the strikethrough plate 9001 is constructed of Plexiglas with an overall dimension of 10.2 cm long by 10.2 cm wide by 3.2 cm tall. A longitudinal channel 9007 that runs the length of the plate is 13 mm deep, 28 mm wide at the top plane of the plate, with lateral walls that slope downward at 65° to a 15 mm wide base. A central test fluid well 9009 is 26 mm long, 24 mm deep, 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying test sample. The test fluid reservoir 9003 has an overall length of 25 mm, width of 15 mm, and depth of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 6 mm apart. The lateral sides of the reservoir bow outward at a radius 9012 of 14 mm bounded by the overall width 2013 of 15 mm. Two wells 9002 (80.5 mm long×24.5 mm wide×25 mm deep) located outboard of the lateral channel, are filled with lead shot (or equivalent) to adjust the overall mass of the plate to provide a constraining pressure of 0.25 psi (17.6 g/cm²) to the test area. Electrodes 9004 are embedded in the plate 9001, connecting the exterior banana jacks 9006 to the inside wall 9005 of the fluid reservoir 9003. A circuit interval timer is plugged into the jacks 9006, and monitors the impedance between the two electrodes 9004, and measures the time from introduction of the AMF into reservoir 9003 until the AMF drains from the reservoir. The timer has a resolution of 0.01 sec.

For the rewet portion of the test, the pressure applied to the test sample is 1.0 psi. The rewet weight is constructed such that the dimensions of the bottom face of the weight match the dimensions of the strikethrough plate, and the total mass required is calculated to give a pressure of 1.0 psi over the bottom face of the weight. Thus, the bottom face of the weight is 10.2 cm long by 10.2 cm wide, and constructed of a flat, smooth rigid material (e.g., stainless steel) to give a mass of 7.31 kg.

For each test sample, seven plies of filter paper cut to 150 mm diameter are used as the rewet substrate. The filter paper is conditioned at 23° C.±2° C. and 50%±2% relative humidity for at least 2 hours prior to testing. A suitable filter paper has a basis weight of about 74 gsm, a thickness of about 157 microns with medium porosity, and is available from VWR International as grade 413.

Test samples are removed from all packaging using care not to press down or pull on the products while handling. No attempt is made to smooth out wrinkles. The test samples are conditioned at 23° C.±2° C. and 50%±2% relative humidity for at least 2 hours prior to testing. Determine the dose location as follows. For symmetrical samples (i.e., the front of the sample is the same shape and size as the back of the sample when laterally divided along the midpoint of the longitudinal axis of the sample), the dose location is the intersection of the midpoints of the longitudinal axis and lateral axis of the sample. For asymmetrical samples (i.e., the front of the sample is not the same shape and size as the back of the sample when laterally divided along the midpoint of the longitudinal axis of the sample), the dose location is the intersection of the midpoint of the longitudinal axis of the sample and a lateral axis positioned at the midpoint of the sample's wings.

The required mass of the strikethrough plate must be calculated for the specific dimensions of the test sample such that a constraining pressure of 0.25 psi is applied. Measure and record the lateral width of the core at the dose location to the nearest 0.1 cm. The required mass of the strikethrough plate is calculated as the core width multiplied by the length of the strikethrough plate (10.2 cm) multiplied by 17.6 g/cm² and recorded to the nearest 0.1 g. Add lead shot (or equivalent) to the wells 9002 in the strikethrough plate to achieve the calculated mass.

Connect the electronic circuit interval timer to the strikethrough plate 9001 and zero the timer. Place the test sample onto a flat, horizontal surface with the body side facing up. Gently place the strikethrough plate 9001 onto the center of the test sample ensuring that the “H” shaped reservoir 9003 is centered over the predetermined dose location.

Using a mechanical pipette, accurately pipette 3.00 mL±0.05 mL of AMF into the test fluid reservoir 9003. The fluid is dispensed, without splashing, along the molded lip of the bottom of the reservoir 9003 within a period of 3 seconds or less. Immediately after the fluid has been acquired, record the acquisition time to the nearest 0.01 seconds and start a 5 minute timer. In like fashion, apply a second and third dose of AMF into the test fluid reservoir, with a 5 minute wait time between each dose. Record the acquisition times to the nearest 0.001 seconds. Immediately after the 3^{rd} dose of AMF has been acquired, start a 5 minute timer and prepare the filter papers for the rewet portion of the test.

Obtain the mass of 7 plies of the filter paper and record as Dry Massrₚ to the nearest 0.001 grams. When 5 minutes have elapsed after the third acquisition, gently remove the strikethrough plate from the test sample and set aside. Place the 7 plies of pre-weighed filter paper onto the test sample, centering the stack over the dosing location. Now place the rewet weight centered over the top of the filter papers and start a 15 second timer. As soon as 15 seconds have elapsed, gently remove the rewet weight and set aside. Obtain the mass of the 7 plies of filter paper and record as Wet Mass_{fp} to the nearest 0.001 grams. Subtract the Dry Mass_{fp} from the Wet Mass_{fp} and report as Rewet Value to the nearest 0.001 grams. Thoroughly clean the electrodes 9004 and wipe off any residual test fluid from the bottom faces of the strikethrough plate and rewet weight prior to testing the next sample.

Immediately following the rewet portion of the test, proceed to the Stain Size method using the dosed test sample, as described herein.

In like fashion, repeat the entire procedure on ten replicate samples. The reported value is the arithmetic mean of the ten individual recorded measurements for Acquisition Times (first, second and third) to the nearest 0.001 seconds and Rewet Value to the nearest 0.001 grams.

### Stain Size Measurement Method

This method describes how to measure the size of a fluid stain visible on an absorbent article. This procedure is performed on test samples immediately after they have been dosed with test liquid according to a separate method, as described herein (e.g., the Repetitive Acquisition and Rewet method). The resultant test samples are photographed under controlled conditions. Each photographic image is then analyzed using image analysis software to obtain measurements of the size of the resulting visible stain. All measurements are performed at constant temperature (23° C.±2° C.) and relative humidity (50%±2%).

The test sample along with a calibrated ruler (traceable to NIST or equivalent) are laid horizontally flat on a matte black background inside a light box that provides stable uniform lighting evenly across the entire base of the light box. A suitable light box is the Sanoto MK50 (Sanoto, Guangdong, China), or equivalent, which provides an illumination of 5500 LUX at a color temperature of 5500K. A Digital Single-Lens Reflex (DSLR) camera with manual setting controls (e.g., a Nikon D40X available from Nikon Inc., Tokyo, Japan, or equivalent) is mounted directly above an opening in the top of the light box so that the entire article and ruler are visible within the camera's field of view.

Using a standard 18% gray card (e.g., Munsell 18% Reflectance (Gray) Neutral Patch/Kodak Gray Card R-27, available from X-Rite; Grand Rapids, MI, or equivalent) the camera's white balance is custom set for the lighting conditions inside the light box. The camera's manual settings are set so that the image is properly exposed such that there is no signal clipping in any of the color channels. Suitable settings might be an aperture setting of f/11, an ISO setting of 400, and a shutter speed setting of 1/400 sec. At a focal length of 35 mm the camera is mounted approximately 14 inches above the article. The image is properly focused, captured, and saved as a JPEG file. The resulting image must contain the entire test sample and distance scale at a minimum resolution of 15 pixels/mm.

To analyze the image, transfer it onto a computer running an image analysis software (a suitable software is MATLAB, available from The Mathworks, Inc, Natick, MA, or equivalent). The image resolution is calibrated using the calibrated distance scale in the image to determine the number of pixels per millimeter. The image is analyzed by manually drawing the region of interest (ROI) boundary around the visibly discernable perimeter of the stain created by the previously dosed test liquid. The area of the ROI is calculated and reported as the Overall Stain Area to the nearest 0.01 mm² along with notation as to which method was used to generate the test sample being analyzed (e.g., Repetitive Acquisition and Rewet).

This entire procedure is repeated on all of the replicate test samples generated from the dosing method(s). The reported value is the average of the individual recorded measurements for the Overall Stain Area to the nearest 0.01 mm² along with notation as to which method was used to generate the test samples that were analyzed (e.g., Repetitive Acquisition and Rewet).

### Artificial Menstrual Fluid (AMF) Preparation

The Artificial Menstrual Fluid (AMF) is composed of a mixture of defibrinated sheep blood, a phosphate buffered saline solution and a mucous component. The AMF is prepared such that it has a viscosity between 7.15 to 8.65 centistokes at 23° C.

Viscosity of the AMF is performed using a low viscosity rotary viscometer (a suitable instrument is the Cannon LV-2020 Rotary Viscometer with UL adapter, Cannon Instrument Co., State College, PA, or equivalent). The appropriate size spindle for the viscosity range is selected, and instrument is operated and calibrated as per the manufacturer. Measurements are taken at 23° C.±1° C. and at 60 rpm. Results are reported to the nearest 0.01 centistokes.

Reagents needed for the AMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, available from Sterilized American Laboratories, Inc., Omaha, NE, or equivalent), 10% v/v lactic acid aqueous solution, 10% w/v potassium hydroxide aqueous solution, sodium phosphate dibasic anhydrous (reagent grade), sodium chloride (reagent grade), sodium phosphate monobasic monohydrate (reagent grade) and deionized water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add deionized water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add deionized water to volume. Mix thoroughly. To prepare the phosphate buffered saline solution, add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. To determine the amount of gastric mucin needed to achieve AMF within the target viscosity range (7.15-8.65 centistokes at 23° C.) prepare 3 batches of AMF with varying amounts of gastric mucin in the mucous component, and then interpolate the exact amount needed from a concentration versus viscosity curve with a least squares linear fit through the three points. A successful range of gastric mucin is usually between 38 to 50 grams.

To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5° C. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range. After the 2.5 hours has elapsed, remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1° C.

Allow the temperature of the sheep blood and mucous component to come to 23° C.±1° C. Using a 500 mL graduated cylinder, measure the volume of the entire batch of the previously prepared mucous component and add it to a 1200 mL beaker. Add an equal volume of sheep blood to the beaker and mix thoroughly. Using the viscosity method previously described, ensure the viscosity of the AMF is between 7.15-8.65 centistokes. If not the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### Dry MD Standard 3 Point Bend

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry test specimens. The intention of this method is to mimic deformation created in the x-y plane by a wearer of an absorbent article during normal use. All testing is performed in a room controlled at 23° C.±3° C. and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be move horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place the bar of the top fixture is parallel to and aligned front to back with the bars of the bottom fixture and is centered between the bars if the bottom fixture. Both fixtures include an integral adapter appropriate to fit the respective position on the universal test frame and lock into position such that the bars are orthogonal to the motion of the crossbeam of the test frame.

The gap (“span”) between the bars of the lower fixture is set to 25 mm+0.5 mm (center of bar to center of bar) with the upper bar centered at the midpoint between the lower bars. Set the gage (bottom of top bar to top of lower bars) to 1.0 cm.

The thickness (“caliper”) of the test specimen is measured using a manually-operated micrometer equipped with a pressure foot capable of exerting a steady pressure of 0.1 psi+0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3+1 mm/s until the full weight of the pressure is exerted onto the specimen. After 5 seconds elapse, the thickness is recorded as caliper to the nearest 0.01 mm.

The absorbent article samples are conditioned at 23° C.±3° C. and 50%±2% relative humidity two hours prior to testing. Dry test specimens are taken from an area of the sample that is free from any seams and residua of folds or wrinkles, and ideally from the center of pad (intersection of longitudinal and lateral midlines). The dry specimens are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, and marking the body-facing surface. The thickness of the test specimen is measured, as described herein, and recorded as dry specimen caliper to the nearest 0.01 mm. The mass of the test specimen is obtained and recorded as dry mass to the nearest 0.001 grams. The basis weight of the test specimen is calculated by dividing the mass (g) by the area (0.002581 m²) and recorded as dry specimen basis weight to the nearest 0.01 g/m². The bulk density of the test specimen is calculated by dividing the specimen basis weight (g/m²) by the specimen thickness (mm), then dividing the quotient by 1000, and recorded as dry specimen density to the nearest 0.01 g/cm³. In like fashion, five replicate dry test specimens are prepared.

The universal test frame is programed for a flexural bend test with the following settings. The motion of the crosshead is initiated such that the top fixture moves down with respect to the lower fixture at a rate of 1.0 mm/sec until the upper bar touches the top surface of the specimen with a nominal force of 0.02 N, then the crosshead continues to move down for an additional 12 mm. The crosshead is then immediately returned to the original gage at a rate of 1.0 mm/s. Force (N) and displacement (mm) data are continuously collected at 100 Hz throughout the test.

A dry test specimen is loaded onto the fixture such that it spans the two lower bars and is centered beneath the upper bar, with its sides parallel to the bars. For MD bending, the MD direction of the test specimen is perpendicular to the length of the 3 bars. The test is started and force and displacement data is continuously collected for the duration of the test.

A graph of force (N) versus displacement (mm) is constructed. From the graph, the maximum peak force is determined and recorded as dry MD peak load to the nearest 0.01 N. The maximum slope of the curve between the initial force and maximum force (during the loading portion of the curve) is calculated and recorded to the nearest 0.1 unit. The modulus is calculated using the following equation and recorded as dry MD modulus to the nearest 0.001 N/mm².

$$Modulus ( N / {mm}^{2} ) = ( Slope \times ( {Span}^{3} ) ) / ( 4 \times specimen width \times ( specimen {caliper}^{3} ) )$$

Bending stiffness is calculated as using the following equation and recorded as dry MD bending stiffness to the nearest 0.1 N mm².

$$Bending stiffness ( N {mm}^{2} ) = Modulus \times Moment of Inertia where Moment of Inertia ( {mm}^{4} ) = ( specimen width \times ( specimen {caliper}^{3} ) ) / 12$$

In like fashion, the procedure is repeated for all five replicates of the dry test specimens. The arithmetic mean among the five replicate dry test specimens is calculated for each of the parameters and reported as Dry Specimen Caliper to the nearest 0.01 mm, Dry Specimen Basis Weight to the nearest 0.01 g/m², Dry Specimen Density to the nearest 0.001 g/cm³, Dry MD Peak Load to the nearest 0.01 N, Dry MD Modulus to the nearest 0.001 N/mm², and Dry MD Bending Stiffness to the nearest N mm².

### Wet Bunch Compression Test

The Wet Bunch Compression test method measures the force versus displacement behavior across five cycles of load application (“compression”) and load removal (“recovery”) of a wetted absorbent article test sample that has been intentionally “bunched”, using a universal constant rate of extension test frame (a suitable instrument is the MTS Alliance using TestSuite software, as available from MTS Systems Corp., Eden Prairie, MN, or equivalent) equipped with a load cell for which the forces measured are within 1% to 99% of the limit of the cell. The test is executed on test specimens that are dosed with a specified amount of test fluid, as described herein. The intention of this method is to mimic the deformation created in the z-plane of the crotch region of an absorbent article, or components thereof, as it is worn by the wearer during sit-stand movements. All testing is performed in a room controlled at 23° C.±3° C. and 50%±2% relative humidity.

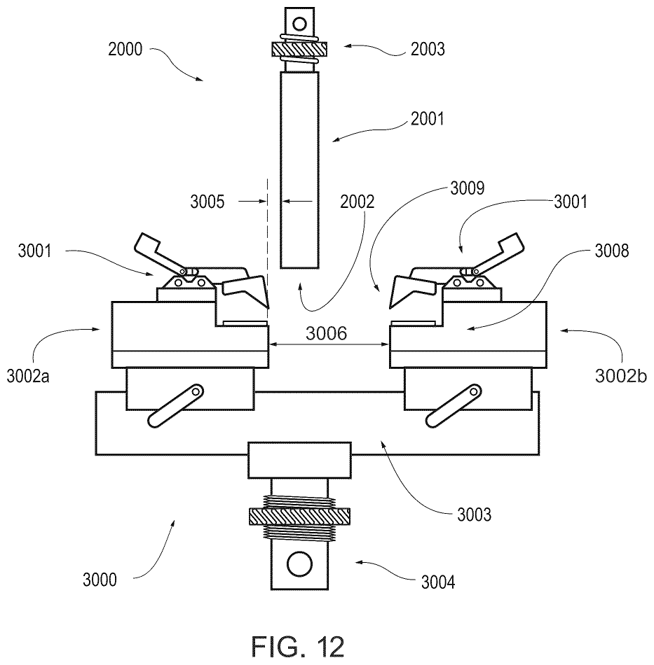

The test apparatus is depicted in FIGS. 12-14. The bottom stationary fixture 3000 consists of two matching sample clamps 3001 each 100 mm wide, each mounted on its own movable platform 3002a, 3002b. The clamp has a “knife edge” 3009 that is 110 mm long, which clamps against a 1 mm thick hard rubber face 3008. When closed, the clamps are flush with the interior side of its respective platform. The clamps are aligned such that they hold an un-bunched specimen horizontal and orthogonal to the pull axis of the tensile tester. The platforms are mounted on a rail 3003 which allows them to be moved horizontally left to right and locked into position. The rail has an adapter 3004 compatible with the mount of the tensile tester capable of securing the platform horizontally and orthogonal to the pull axis of the tensile tester. The upper fixture 2000 is a cylindrical plunger 2001 having an overall length of 70 mm with a diameter of 25.0 mm. The contact surface 2002 is flat with no curvature. The plunger 2001 has an adapter 2003 compatible with the mount on the load cell capable of securing the plunger orthogonal to the pull axis of the tensile tester.

Test samples are conditioned at 23° C.±3° C. and 50%±2% relative humidity for at least 2 hours before testing. Prepare the test specimen as follows. When testing an intact absorbent article, remove the release paper from any panty fastening adhesive on the garment facing side of the article, if present. Lightly apply talc powder to the adhesive to mitigate any tackiness. If there are cuffs, excise them with scissors so as not to disturb the topsheet or any other underlying layers of the article. Place the article, body facing surface up, on a benchtop. On the article, mark the intersection of the longitudinal midline and the lateral midline. Using a rectangular cutting die or equivalent cutting means, cut a specimen 100 mm in the longitudinal direction by 80 mm in the lateral direction, centered at the intersection of the midlines. When testing a material layer or layered components from an absorbent article, place the material layer or layered components on a benchtop and orient as it would be integrated into a finished article, i.e., identify the body facing surface and the lateral and longitudinal axis. Using a rectangular cutting die, or equivalent cutting means, cut a specimen 100 mm in the longitudinal direction by 80 mm in the lateral direction, centered at the intersection of the midlines. Measure the mass of the specimen and record to the nearest 0.001 grams. Calculate the basis weight of the specimen by dividing the mass (g) by the area (0.008 m²) and record as basis weight to the nearest 1 g/m². The test specimen is further prepared by applying a single dose of 10% w/v saline solution, as follows. The saline solution is prepared by adding 100 g of reagent grade NaCl to a 1 L volumetric flask and adding distilled water to the fill line. The volume of the liquid dose applied to the test specimen is 7 mL. The liquid dose is added using a calibrated Eppendorf-type pipettor, spreading the fluid over the complete body facing surface of the specimen within a period of approximately 3 sec. The wet specimen is tested 10.0 min±0.1 min after the dose is applied.

Program the tensile tester to zero the load cell, then lower the upper fixture at 2.00 mm/sec until the contact surface of the plunger touches the specimen and 0.02 N is read at the load cell. Zero the crosshead. Program the system to lower the crosshead 15.00 mm at 2.00 mm/sec then immediately raise the crosshead 15.00 mm at 2.00 mm/sec. This cycle is repeated for a total of five cycles, with no delay between cycles. Data is collected at 50 Hz during all compression/decompression cycles.

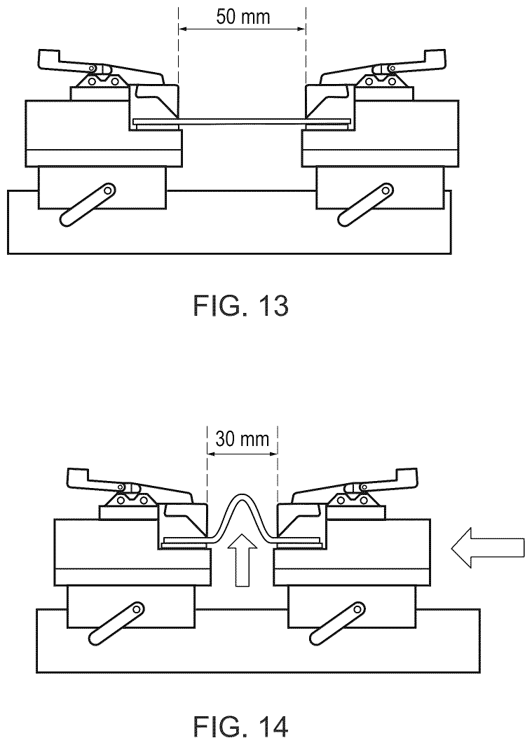

Position the left platform 3002a 2.5 mm from the side of the upper plunger (distance 3005). Lock the left platform into place. This platform 3002a will remain stationary throughout the experiment. Align the right platform 3002b 50.0 mm from the stationary clamp (distance 3006). Raise the upper probe 2001 such that it will not interfere with loading the specimen. Open both clamps 3001. Referring to FIG. 13, place the dry specimen with its longitudinal edges (i.e., the 100 mm long edges) within the clamps. With the dry specimen laterally centered, securely fasten both edges in the clamps. Referring to FIG. 14, move the right platform 3002b toward the stationary platform 3002a a distance 30.0 mm. Allow the dry specimen to bow upward as the movable platform is positioned. Now manually lower the probe 2001 until the bottom surface is approximately 1 cm above the top of the bowed specimen.

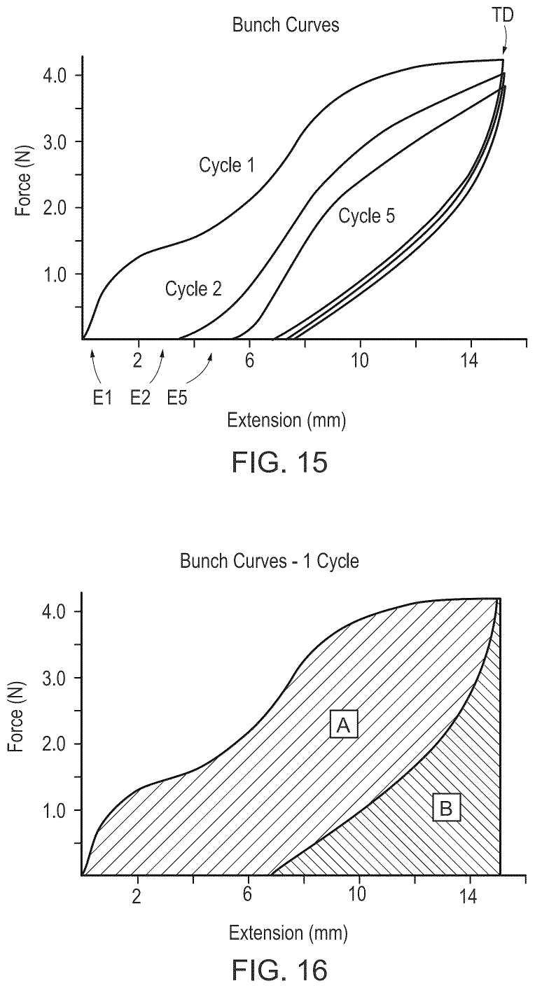

Start the test and continuously collect force (N) versus displacement (mm) data for all five cycles. Construct a graph of force (N) versus displacement (mm) separately for all cycles. A representative curve is shown in FIG. 15. From the curve, determine the Wet Maximum Compression Force for each Cycle to the nearest 0.01 N, then multiply by 101.97 and record to the nearest 1 gram-force. Calculate the Wet % Recovery between the First and Second cycle as (TD−E2)/(TD−E1)*100 where TD is the total displacement and E2 is the extension on the second compression curve that exceeds 0.02 N, and record to the nearest 0.01%. In like fashion calculate the Wet % Recovery between the First Cycle and other cycles as (TD−E1)/(TD−E1)*100 and record to the nearest 0.01%. Referring to FIG. 16, calculate the Wet Energy of Compression for Cycle 1 as the area under the compression curve (i.e., area A+B) and record to the nearest 0.1 N-mm. Calculate the Wet Energy Loss from Cycle 1 as the area between the compression and decompression curves (i.e., Area A) and record to the nearest 0.1 N-mm. Calculate the Wet Energy of Recovery for Cycle 1 as the area under the decompression curve (i.e., Area B) and report to the nearest 0.1 N-mm. In like fashion calculate the Wet Energy of Compression (N-mm), Wet Energy Loss (N-mm) and Wet Energy of Recovery (N-mm) for each of the other cycles and record to the nearest 0.1 N-mm.

The overall procedure is now repeated for a total of five replicate wet test specimens, reporting results for each of the five cycles as the arithmetic mean among the five wet replicates for Wet Maximum Compression Force to the nearest 1 gram-force, Wet Energy of Compression to the nearest 0.1 N-mm, Wet Energy Loss to the nearest 0.1 N-mm, and Wet Energy of Recovery to the nearest 0.1 N-mm.

New Artificial Menstrual Fluid (nAMF) Preparation

This formulation of new Artificial Menstrual Fluid (nAMF) is composed of a mixture of defibrinated sheep blood, a phosphate buffered saline solution and a mucous component. The nAMF is prepared such that it has a viscosity between 7.40 to 9.00 centipoise at 23° C.

Viscosity of the nAMF is performed using a low viscosity rotary viscometer (a suitable instrument is the Brookfield DV2T fitted with a Brookfield UL adapter, available from AMETEK Brookfield, Middleboro, MA, or equivalent). The appropriate size spindle for the viscosity range is selected, and the instrument is operated and calibrated as per the manufacturer. Measurements are taken at 23° C.±1° C. and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, sterilized, available from American Laboratories, Inc., Omaha, NE, or equivalent), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare about 200 mL of phosphate buffered saline solution, add 49.50 g±0.10 g of Solution A and 157.50 g+0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, NC, or equivalent. After the final mixing step, measure and record the viscosity of the mucous component to the nearest 0.01 centipoise at 23° C.±1° C. and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1° C. To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1° C. and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1° C. Any unused portion is discarded after testing is complete.

### Z-Compression Method

The Z-compression method measures the compression behavior along the z-direction of a test specimen, on a Constant Rate of Extension (CRE) universal mechanical test system using a load cell for which the forces measured are within 1% to 99% of the limit of the cell (preferably 100 N). A suitable instrument is the MTS Alliance using TestSuite Software, as available from MTS Systems Corp., Eden Prairie, MN, or equivalent. All testing is performed in a room controlled at 23° C.±3° C. and 50%±2% relative humidity.

The upper and lower fixtures of the test system are circular parallel plate compression platens made of stainless steel. The platen mounted on the moveable CRE fixture has a diameter of 40 mm, and the platen mounted on the stationary CRE fixture has a diameter>40 mm. Both platens have adapters compatible with the mounts of the CRE test machine, capable of securing the platens with their opposing surfaces lying along parallel planes that are orthogonal to the motion of the crossbeam of the CRE test machine.

The absorbent article samples are conditioned at 23° C.±3° C. and 50%±2% relative humidity two hours prior to testing. Remove the test sample from its outer wrapper, then remove the protective cover/release paper from the panty fastening adhesive on the garment facing side of the sample. Lightly apply talc powder to the adhesive to mitigate any tackiness. To obtain a test specimen for measurement, a circular die with a diameter of 40 mm is used. If wings are present on the absorbent article test sample, the test specimen is obtained by centering the circular die over the intersection of the longitudinal midpoint and a lateral line that is centered within the region where the wings are located. If wings are not present, the test specimen is obtained by centering the circular die over the intersection of the longitudinal and lateral midpoints of the absorbent article test sample. In like fashion, five replicate test specimens are prepared from five absorbent article test samples.

Prepare the universal test frame for a compression test to measure force and distance for one cycle of loading (compression) and unloading (recovery) as follows. The crosshead motion is programmed such that the upper platen moves down with respect to the lower platen at a rate of 0.2 mm/s until an endpoint load of 8.66 N (6.9 kPa) is reached, then the crosshead immediately returned to the original gauge (platen separation distance).

Execute the test as follows. Move the platens such that the initial distance between contact surfaces of the platens (gauge) is 25 mm, then zero the crosshead and load cell. Place the test specimen, with the wearer-facing surface upward, onto the bottom platen with the center of the test specimen centered below the upper platen. Manually adjust the position of the upper platen such that its contact surface is about 1 mm above the upper surface of the test specimen. Start the test and continuously collect force (N) and displacement (mm) data at a rate of 100 Hz.

Construct a graph of force (N) versus thickness (mm), across the array of data collected for the entire cycle. To note, at each datapoint, thickness is the original gauge (25 mm) minus the crosshead position (mm). From the resulting force (N) vs thickness (mm) curve, calculate the area under the loading (compression) portion of the curve from the initial thickness to the minimum thickness, and record as energy of compression to the nearest 0.001 N*mm.

In like fashion, the procedure is repeated for all five replicate test specimens. The arithmetic mean across all five replicates is calculated and reported as Energy of Compression to the nearest 0.001 N*mm.

### Peak Load and Elongation Method

The peak load and elongation of a test specimen are measured as the specimen is pulled to failure using a universal constant rate of extension test frame. Measurements are made on test specimens prepared from the machine direction (MD) and the cross direction (CD) of the test material. All measurements are performed in a laboratory maintained at 23° C.±2° C. and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 35 mm wide. The fixtures are installed on the universal test frame and mounted such that they are horizontally and vertically aligned with one another.

Measurements are made on both MD (machine direction) and CD (cross direction) test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained as follows. The MD test specimen is obtained from an area on the test material that is free of any residual of folds or wrinkles. The MD test specimen is cut to a width of 1 inch (25.4 mm) with a length that can accommodate a test span of 51 mm. The long side of the MD test specimen is parallel to the longitudinal axis of the test material as it would appear in an absorbent article. In like fashion, prepare five replicate MD test specimens. The CD test specimen is also cut to a width of 1 inch (25.4 mm) with a length that can accommodate a test span of 51 mm. The long side of the CD test specimen is parallel to the lateral axis of the test material as it would appear in an absorbent article. In like fashion, prepare five replicate CD test specimens.

The universal test frame is prepared for a constant rate of extension tensile test to failure, with slack compensation and gage length adjustment as follows. The initial grip to grip separation distance is set to a nominal gage length (Lₙₒₘᵢₙₐₗ) of 51 mm and then the crosshead is zeroed. The test frame is programmed to move the grips closer together by an intentional slack of 1 mm to ensure no pretension force exists on the test specimen at the onset of the test. (During this motion, the specimen will become slack between the tensile grips.) Next, the grips will move apart at a slack speed of 1 mm/s until the slack preload of 0.10 N is exceeded. At this point, the following are true. 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). The grips will then continue to move apart at a test speed of 254 mm/min until test specimen failure. The grips then return to the nominal gage length.

The test is executed by inserting the MD test specimen into the grips such that the long axis of the specimen is parallel and centered with the motion of the crosshead. The test is started and time, force and displacement data are continuously collected throughout the test at a data acquisition rate of 100 Hz.

A graph of force (N) versus displacement (mm) is constructed. The maximum force value (N) is recorded as MD peak load to the nearest 0.01 N. The crosshead position corresponding to the maximum force value is recorded as Lₚₑₐₖ to the nearest 0.01 mm. The MD percent elongation at the peak load is calculated using the following equation and recorded to the nearest 1 percent.

$$\% Elongation at Peak = ( ( {L}_{p e a k} - {L}_{o} ) / {L}_{o} ) * 100$$

where Lₒ was previously defined as Lₙₒₘᵢₙₐₗ+L_{slack}. The overall procedure is now repeated until all five MD test specimens have been tested. The arithmetic mean among the five replicate test specimens is calculated for each of the recorded parameters and reported as MD Peak Load to the nearest 0.01 N and MD % Elongation at Peak to the nearest 1 percent.

In like fashion, the entire procedure is repeated for the five CD test specimens, and the arithmetic mean among the five replicate test specimens is calculated and reported as CD Peak Load the nearest 0.01 N and CD % Elongation at Peak to the nearest 1 percent.

### Bending Length

For the Bending Length testing; compendial method WSP 90.5 (05) is followed.

### EXAMPLES AND DATA

| | Inventive | Inventive | Inventive | Inventive | Inventive |
|---|---|---|---|---|---|
| | Sample 1 | Sample 2 | Sample 3 | Sample 4 | Sample 5 |
| Basis Weight (g/m²) | 71.68 | 65.61 | 64.98 | 57.54 | 62.00 |
| Caliper 0.5 kPa (mm) | 1.85 | 1.88 | 1.88 | 1.79 | 1.87 |
| CALIPER Factor | 0.26 | 0.29 | 0.29 | 0.31 | 0.30 |
| (mm/10 gsm) | | | | | |
| MD/CD Peak Load ratio | 0.65 | 0.69 | 0.84 | 0.92 | 0.94 |
| CD Peak Load (N) | 45.61 | 7.17 | 9.85 | 7.20 | 36.70 |
| CD Peak Load/Basis | 0.64 | 0.11 | 0.15 | 0.13 | 0.59 |
| Weight (N * gsm) | | | | | |
| CD Elongation (%) | 100.86 | 33.56 | 41.50 | 47.95 | 104.50 |
| CD Elongation/Basis | 1.41 | 0.51 | 0.64 | 0.83 | 1.69 |
| Weight (%/gsm) | | | | | |
| CD Bending length | 3.07 | 3.07 | 3.11 | 1.53 | — |
| flexural rigidity (mN*cm) | | | | | |
| CD Bending length | 0.04 | 0.05 | 0.05 | 0.03 | — |
| flexural rigidity/Basis | | | | | |
| Weight (mN*cm/gsm) | | | | | |
| MD Peak Load (N) | 29.46 | 4.98 | 8.27 | 6.59 | 34.40 |
| MD Peak Load/Basis | 0.41 | 0.08 | 0.13 | 0.11 | 0.55 |
| Weight (N *gsm) | | | | | |
| MD Elongation (%) | 100.86 | 59.76 | 67.43 | 57.32 | 65.60 |
| MD Elongation/Basis | 1.41 | 0.91 | 1.04 | 1.00 | 1.06 |
| Weight (%/gsm) | | | | | |
| MD Bending length | 6.31 | 3.01 | 4.19 | 2.35 | — |
| flexural rigidity (mN*cm) | | | | | |
| MD Bending length | 0.09 | 0.05 | 0.06 | 0.04 | — |
| flexural rigidity/Basis | | | | | |
| Weigh (mN*cm/gsm) | | | | | |
| Air Permeability | 203.93 | 150.87 | 147.60 | 221.00 | 99.50 |
| (m³/m²/min) | | | | | |
| Average pore size (um) | 299 | 120 | — | — | — |

Inventive Sample 1—basis weight of 71.68 gsm having 25 percent by weight viscose cellulose fibers having a 1.3 dtex; 75 percent by weight bi-component fibers having a first component polyethylene terephthalate and polyethylene in a core-sheath configuration where the polyethylene is the sheath.
Inventive Sample 2—basis weight of 65.61 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath.
Inventive Sample 3—basis weight of 64.98 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath.
Inventive Sample 4—basis weight of 57.54 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath.
Inventive Sample 5—basis weight of 62.00 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath.

| | | | |
|---|---|---|---|
| Dry MD 3 Point Bend | 2.9 | 2.8 | 2.8 |
| Caliper (mm) | | | |
| Z-Compression | 3.9 | 3.5 | 3.0 |
| Compression Energy (N*mm) | | | |
| Dry MD 3 Point Bend | 37.0 | 18.5 | 24.1 |
| Stiffness (N*m²) | | | |
| Wet Bunch Compression % | 58 | 44 | 48 |
| Recovery 1^{st} to 5^{th} cycle | | | |
| Wet Bunch Compression | 1.8 | 1.9 | 1.2 |
| Energy of Recovery 5th cycle | | | |
| (N*m²) | | | |
| RATR (3 ml gush) 1, 2, 3 | 6.7, 18.8, 32.8 | 12.2, 40.9, 70.1 | 13.5, 42.3, 71.7 |
| Acquisition Time (s) | | | |
| ATRM IFF (mg) | 55.0 | 26.4 | 38.8 |
| ATRM SFF (mg) | 23.2 | 13.2 | 34.3 |
| ATRM Rewet (mg) | 232.1 | 142.8 | 636.7 |
| Dry MD 3 Point Bend | 2.9 | 2.8 | 2.8 |
| Caliper (mm) | | | |

Inventive Products 1, 2, and 3 all contain the same topsheet. The topsheet is a nonwoven topsheet having a basis weight of 24 gsm and being an air-through bonded, nonwoven. The air-through bonded nonwoven comprised bi-component fibers, where polyethylene terephthalate and polyethylene were in a core-sheath configuration with polyethylene as the sheath. The fibers comprise a 4.4 dtex. The topsheet comprised 60% hydrophilic fibers and 40% hydrophobic fibers by weight of the fibers.

Inventive Products 1, 2, and 3 all contain the same backsheet. The backsheet is 12 gsm polypropylene film, available from RKW.

Inventive Products 1, 2, and 3 all contain the same core. The absorbent core is an airlaid absorbent core comprising pulp fibers, absorbent gelling material, and bico fibers, having a basis weight of 150 gsm available from Glatfelter, York, Pa., USA have 22 gsm of absorbent gelling material.

Inventive Product 1 additionally contained a secondary layer between the topsheet and core, having a basis weight of 71.68 gsm having 25 percent by weight viscose cellulose fibers having a 1.3 dtex; 75 percent by weight bi-component fibers having a first component polyethylene terephthalate and polyethylene in a core-sheath configuration where the polyethylene is the sheath. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Inventive Product 2 additionally contained a secondary layer between the topsheet and core, having a basis weight of 65.61 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Inventive Product 3 additionally contained a secondary layer between the topsheet and core, having a basis weight of 64.98 gsm having 25 percent by weight viscose cellulose fibers having a 0.9 dtex; 45 percent by weight tri-lobal polypropylene fibers having a 1.0 dtex; 30 percent by weight bi-component fibers having a first component polyethylene terephthalate and polyethylene in a core-sheath configuration where the polyethylene is the sheath.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

### EXAMPLES/PARAGRAPHS

Paragraph 1. A fluid management layer comprising:
a. a nonwoven having a basis weight of from about 40 gsm to about 75 gsm,
b. from about 15 to about 35 weight percent of cellulosic fibers,
c. from about 65 to about 85 weight percent of bonding fibers,
   wherein the fluid management layer has a caliper factor of from about 0.26 to about 0.35, and wherein the cellulosic fibers and the bonding fibers have a decitex below about 2.
      Paragraph 2. The fluid management layer of paragraph 1, further comprises integrated stitches at a stitch density of between 90 and 220 punches per square centimeter.
      Paragraph 3. The fluid management layer of paragraph 1-2 wherein the stitch direction is selected from a top stitch direction, a bottom stitch direction, and combinations thereof.
      Paragraph 4. The fluid management layer of Paragraph 1-3, wherein the fiber management layer has an MD:CD Peak Load ratio from about 0.5 to about 1.75.
      Paragraph 5. The fluid management layer of Paragraph 1-4, further comprising divider fibers having a decitex of from about 0.5 to about 2.
      Paragraph 6. The fluid management layer of Paragraph 1-5, wherein the divider fibers are selected from polypropylene, polyethylene terephthalate, bicomponent polyethylene, bicomponent polypropylene, bicomponent polyethylene terephthalate and combinations thereof.
      Paragraph 7. The fluid management layer of Paragraph 1-6, wherein the divider fibers are non-cylindrical polypropylene.
      Paragraph 8. The fluid management layer of Paragraph 1-7, wherein the cellulosic fibers are selected from cotton, rayon, viscose, lyocell, natural cellulose, regenerated cellulose and combinations thereof.
      Paragraph 9. The fluid management system of Paragraph 1-8, wherein the cellulosic fibers are viscose.
      Paragraph 10. The fluid management layer of Paragraph 1-9, wherein the bonding fibers are selected from Bicomponent polyethylene terephthalate/polyethylene, combinations of polyethylene, polypropylene, polyethylene terephthalate, Co-polyethylene terephthalate and combinations thereof.
      Paragraph 11. The fluid management layer of Paragraph 1-10, wherein the bonding fibers are polyethylene terephthalate/polyethylene wherein the core is polyethylene terephthalate and the sheath is polyethylene.
      Paragraph 12. The fluid management layer of Paragraph 1-11, wherein the bonding fibers comprise bicomponent fibers.
      Paragraph 13. The fluid management layer of Paragraph 1-12, wherein the bonding fibers further comprise non-cylindrical polymeric fibers.
      Paragraph 14. The fluid management layer of Paragraph 1-13, wherein cellulosic fibers have a decitex of from about 0.5 to about 1.7.
      Paragraph 15. The fluid management layer of Paragraph 1-14, wherein the bonding fibers have a decitex of from about 1 to about 2.
      Paragraph 16. The fluid management layer of Paragraph 1-15, wherein the fluid management layer has a MD peak load of from about 4 to about 85 Newtons.
      Paragraph 17. The fluid management layer of Paragraph 1-16, wherein the fluid management layer has a CD peak load of from about 4 to about 130 Newtons.
      Paragraph 18. The fluid management layer of Paragraph 1-17 wherein the average pore size is from about 90 to about 330 μm.
      Paragraph 19. The fluid management layer of Paragraph 1-18, wherein the average pore size is from about 100 to about 150 μm.
      Paragraph 20. The fluid management layer of Paragraph 1-19, wherein the fibers are from about 10 to about 120 mm in length.
      Paragraph 21. The fluid management layer of Paragraph 1-20, wherein the fibers are from about 24 to about 95 mm in length.
      Paragraph 22. The fluid management layer of Paragraph 1-21, wherein the fibers are from about 36 to about 75 mm in length.
      Paragraph 23. The fluid management layer of Paragraph 1-22, wherein the fibers length is selected from a same length, a different length, or combinations thereof.
      Paragraph 24. A disposable absorbent article comprising a topsheet, a backsheet, an absorbent core disposed between the topsheet and the backsheet, and a fluid management disposed between the topsheet and the absorbent core wherein the fluid management layer comprises a nonwoven having a basis weight of from about 40 gsm to about 75 gsm, from about 15 to about 35 weight percent of cellulosic fibers, from about 65 to about 85 weight percent of bonding fibers, wherein the fluid management layer has a caliper factor of from about 0.26 to about 0.35, and wherein the cellulosic fibers and the polymeric fibers have a decitex below about 2.
      Paragraph 25. The absorbent article of Paragraph 24, wherein the absorbent article has a Z-compression Compression Energy of from about 2.6 N·mm to about 4.0 N·mm, a 3 point MD bend dry bending stiffness of from about 15 N·mm\hat{ }2 and about 40 N·mm\hat{ }2, and a wet bunch compression % recovery of greater than about 40%.
      Paragraph 26. The absorbent article of Paragraph 24-25, wherein the topsheet comprises an anti-stick agent on the wearer facing side of the topsheet.
      Paragraph 27. The absorbent article of Paragraph 24-26, wherein the anti-stick agent comprises a propylene glycol material.
      Paragraph 28. The absorbent article of Paragraph 24-27, wherein the propylene glycol material is polypropylene glycol.
      Paragraph 29. The absorbent article of Paragraph 24-28, further comprising divider fibers.
      Paragraph 30. The absorbent article of Paragraph 24-29, wherein the divider fibers are selected from polypropylene, polyethylene terephthalate, bicomponent polyethylene, bicomponent polypropylene, bicomponent polyethylene terephthalate and combinations thereof.
      Paragraph 31. The absorbent article of Paragraph 24-30, wherein the divider fibers are polypropylene.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fluid management layer comprising:
a nonwoven having a basis weight of from about 40 gsm to about 75 gsm, wherein the nonwoven comprises:
from about 15 to about 35 weight percent of cellulosic fibers,
from about 65 to about 85 weight percent of bonding fibers,
wherein the fluid management layer has a caliper factor of from about 0.26 to about 0.35, and wherein the cellulosic fibers and the bonding fibers have a decitex below about 2.

2. The fluid management layer of claim 1, comprises integrated stitches at a stitch density of between 90 and 220 punches per square centimeter.

3. The fluid management layer of claim 2, wherein the stitch direction is selected from a top stitch direction, a bottom stitch direction, and combinations thereof.

4. The fluid management layer of claim 1, wherein the fiber management layer has an MD:CD Peak Load ratio from about 0.5 to about 1.75.

5. The fluid management layer of claim 1, comprising divider fibers having a decitex of from about 0.5 to about 2.

6. The fluid management layer of claim 5, wherein the divider fibers are selected from polypropylene, polyethylene terephthalate, bicomponent polyethylene, bicomponent polypropylene, bicomponent polyethylene terephthalate and combinations thereof.

7. The fluid management layer of claim 6, wherein the divider fibers are non-cylindrical polypropylene.

8. The fluid management layer of claim 1, wherein the cellulosic fibers are selected from cotton, rayon, viscose, lyocell, natural cellulose, regenerated cellulose and combinations thereof.

9. The fluid management layer of claim 1, wherein the bonding fibers are selected from Bicomponent polyethylene terephthalate/polyethylene, combinations of polyethylene, polypropylene, polyethylene terephthalate, Co-polyethylene terephthalate and combinations thereof.

10. The fluid management layer of claim 1, wherein cellulosic fibers have a decitex of from about 0.5 to about 1.7.

11. The fluid management layer of claim 1, wherein the bonding fibers have a decitex of from about 1 to about 2.

12. The fluid management layer of claim 1, wherein the fluid management layer has a MD peak load of from about 4 to about 85 Newtons, and wherein the fluid management layer has a CD peak load of from about 4 to about 130 Newtons.

13. The fluid management layer of claim 1, wherein the average pore size is from about 90 to about 330 μm.

14. The fluid management layer of claim 1, wherein the fibers are from about 10 to about 120 mm in length.

15. The fluid management layer of claim 14, wherein the fibers are from about 24 to about 95 mm in length.

16. The fluid management layer of claim 1, wherein the fibers length is selected from a same length, a different length, or combinations thereof.

17. A disposable absorbent article comprising a topsheet, a backsheet, an absorbent core disposed between the topsheet and the backsheet, and a fluid management disposed between the topsheet and the absorbent core wherein the fluid management layer comprises a nonwoven having a basis weight of from about 40 gsm to about 75 gsm, from about 15 to about 35 weight percent of cellulosic fibers, from about 65 to about 85 weight percent of bonding fibers, wherein the fluid management layer has a caliper factor of from about 0.26 to about 0.35, and wherein the cellulosic fibers and the polymeric fibers have a decitex below about 2.

18. The absorbent article of claim 17, wherein the absorbent article has a Z-compression compression energy of from about 2.6 N·mm to about 4.0 N·mm, a 3 point MD bend dry bending stiffness of from about 15 N·mm\hat{ }2 and about 40 N·mm\hat{ }2, and a wet bunch compression % recovery of greater than about 40%.

19. The absorbent article of claim 17, comprising divider fibers.

20. The absorbent article of claim 19, wherein the divider fibers are selected from polypropylene, polyethylene terephthalate, bicomponent polyethylene, bicomponent polypropylene, bicomponent polyethylene terephthalate and combinations thereof.
